(19) United States
(12) Patent Application Publication
(10) Pub. No.: US 2014/0187507 A1
(43) Pub. Date: Jul. 3, 2014

(54) TREATMENT OF PARKINSONS DISEASE WITH GLYCOLIPIDS

(71) Applicant: Rutgers, The State University of New Jersey, New Brunswick, NJ (US)

(72) Inventors: Shawn DeFrees, New Brunswick, NJ (US); Robert Ledeen, New Brunswick, NJ (US); Gusheng Wu, New Brunswick, NJ (US)

(21) Appl. No.: 14/108,175

(22) Filed: Dec. 16, 2013

Related U.S. Application Data
(60) Provisional application No. 61/737,581, filed on Dec. 14, 2012.

(51) Int. Cl. C07H 15/26; C07H 15/18; C07H 15/10

(52) U.S. Cl. CPC C07H 15/26; C07H 15/10; C07H 15/18
USPC 514/32; 514/25

(57) ABSTRACT

This invention provides compounds, compositions, and methods for treating Parkinson's disease. In particular, there is provided GM1 ganglioside analogs that are capable of penetrating the blood brain barrier and entering the cytoplasm of neurons. Endogenous GM1 interacts with the with GFRα1/RET, the GDNF receptor complex involved in GDNF signaling which is essential for neuron survival. In neurons that are deficient in endogenous GM1, the GM1 analog compounds of the invention are capable of restoring GDNF signaling thereby preventing neuron cell death.

### CROSS-REFERENCE TO RELATED APPLICATION

This application claims the benefit under 35 U.S.C. §119(e) of U.S. Provisional Application No. 61/737,581, filed Dec. 14, 2012, the entire contents of which is hereby incorporated by reference.

### GOVERNMENT INTERESTS

This invention was made with government support under 5 R01 NS033912 awarded by the National Institutes of Health. The government has certain rights in the invention.

### FIELD OF THE INVENTION

This invention relates to methods of treating Parkinson's disease by administering a suitable glycolipid, ganglioside or ganglioside analog comprising the oligosaccharide moiety of the deficient ganglioside or of a ganglioside downstream of the deficient ganglioside.

### BACKGROUND OF THE INVENTION

Parkinson's disease (PD) is a slowly progressive, degenerative CNS disorder characterized by slow and decreased movement, muscular rigidity, resting tremor, postural instability, cognitive impairment and dementia. The major pathological feature of PD is selective degeneration of dopaminergic neurons in the substantia nigra pars compacta (SNpc) and loss of their terminals in the caudate and putamen. Loss of substantia nigra neurons, which project into the caudate nucleus and putamen, depletes dopamine in these areas. Evidences accumulated in the past indicate that multiple factors, including genetic and environmental ones, contribute to dopaminergic neurodegeneration in this neurodegenerative disease.

A number of mechanisms have been postulated for the dopaminergic neurodegeneration characteristic of PD including a deficiency of glycolipids such as GM1 (Wu et al. (2012) J. Neurosci. Res. 90, 1997-2008.). A decline in GM1 levels in brain tissues is associated with motor decline, an effect that creates dysfunctional dopaminergic neurons and accelerates cell death, in vitro and in vivo (Wu et al. (2012) J. Neurosci. Res. 90, 1997-2008; Wu et al. (2011) J. Neurochem. 116, 714-720). GM1's role in neuron survival and function is linked to multiple cellular processes that require GM1 for normal function such as neurotrophin signaling (Mocchetti et al, (2005) Cell Mol. Life Sci. 62, 2283-2294; Duchemin et al. (2008) J. Neurochem. 104, 1466-1477), and calcium homeostasis (Wu et al. (2009) Proc. Natl. Acad. Sci. USA 106, 10829-10834).

Gangliosides such as GM1 are sialic acid containing glycosphingolipids. Gangliosides are normal components of plasma membranes which are particularly abundant in the nervous system. In humans, gangliosides are most abundant in the gray matter of the brain, particularly in nerve endings. They are believed to be present at receptor sites for neurotransmitters, including acetylcholine, and can also act as specific receptors for other biological macromolecules, including interferon, hormones, viruses, bacterial toxins, and the like.

It has been widely demonstrated that glycosphingolipids, and particularly, gangliosides are able to enhance functional recovery both in the lesioned peripheral nervous system (PNS) and the central nervous system (CNS), through the involvement of specific membrane mechanisms and the interaction with trophic factors, as pointed out from studies in vitro on neuronal cultures (Ferrari, F. et al., Dev. Brain Res. 8:215-221 (1983); Doherty, P. et al., J. Neurochem. 44:1259-1265(1985)); Skaper, S. D. et al., Mol. Neurobiol. 3:173-199 (1989)). Gangliosides have been used for treatment of nervous system disorders, including cerebral ischemic strokes. See, e.g., Mahadik et al., Drug Development Res. 15:337-360(1988); U.S. Pat. Nos. 4,710,490 and 4,347,244; Horowitz, Adv. Exp. Med. and Biol. 174:593-600(1988); Karpiak et al., Adv. Exp. Med. and Biol. 174: 489-497(1984).

As a result, attempts have been made to use gangliosides in the treatment of disorders of the nervous system. This has led to the development of synthetic gangliosides as well as natural ganglioside containing compositions for use in the treatment of disorders of the nervous system (see, U.S. Pat. Nos. 4,476,119; 4,593,091; 4,639,437; 4,707,469; 4,713,374; 4,716,223; 4,849,413; 4,940,694; 5,045,532; 5,135,921; 5,183,807; 5,190,925; 5,210,185; 5,218,094; 5,229,373; 5,260,464; 5,264,424; 5,350,841; 5,424,294; 5,484,775; 5,519,007; 5,521,164; 5,523,294; 5,677,285; 5,792,858; 5,795,869; and 5,849,717).

The biological activity of gangliosides depends upon the particular glycoform, the structure of the ceramide-like backbone of the molecule, including the structure of the fatty acid amide component. Advantages of ganglioside compositions that have structures other than those found in nature include, for example, increased therapeutic half-life due to reduced clearance rate, enhanced bioavailability, and altered bioactivity. Alteration of the structure of a ganglioside can also be used to target the ganglioside to a particular tissue or cell surface receptor that is specific for the altered ganglioside. Variations in the lipid portion of gangliosides can target endothelial cells, promote CNS penetration, dermal absorption, and enhance penetration of cell membranes (e.g., phytosphingomyelin-based gangliosides). The altered ganglioside can also be used as an inhibitor of the receptor, preventing binding of its natural ligand.

The peripheral subcutaneous administration of GM1 to Parkinson's disease patients has slowed the progression of motor function loss when administered for 5 years. However, less than 1% of the administered dose was able to penetrate the brain and the reported improvements were limited to a slowing of motor function loss. A similar improvement in motor function is observed in mouse models of Parkinson's disease when GM1 is administered peripherally; however, no improvement in cognitive function or decline has been reported. There exists a need in the art for ganglioside compounds and methods of treatment which use the compounds to treat the ganglioside deficiency state of Parkinson's disease and the associated loss of cognitive and motor function.

### BRIEF SUMMARY OF THE INVENTION

In various aspects, this invention provides compounds, compositions, and methods for treating Parkinson's disease. In a first aspect, the invention provides methods of treating a subject for Parkinson's disease by administering a compound of the formula (I)-(V):

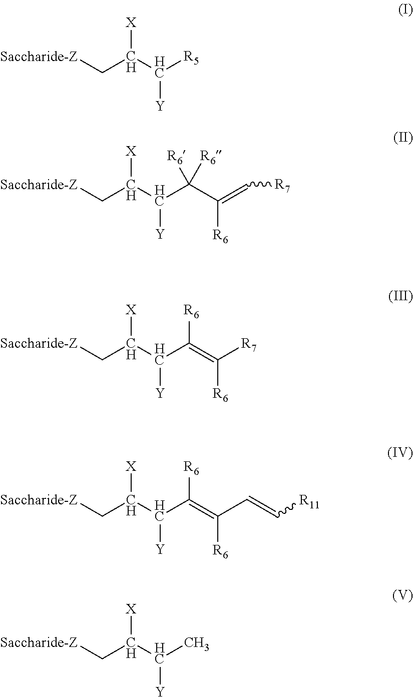

wherein:

### X is H, D, —OR₃, —NR₃R₄, —SR₃ or —CHR₃R₄;

Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;

### Z is O, S or NR₆;

R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and M is O, S or NR₆.

In an embodiment, a ganglioside analog of the present invention can have M and Z independently selected from the group consisting of O, NR₆ or S, and Y is selected from H, —OR⁷, SR⁷, NR⁷R⁸, substituted or unsubstituted alkyl, substituted or unsubstituted heteroalkyl, substituted or unsubstituted aryl, substituted or unsubstituted heteroaryl, and substituted or unsubstituted heterocycloalkyl. Further still, a novel ganglioside of the invention can have R⁵, R⁶, R⁷ and R⁸ independently selected from H, D, substituted or unsubstituted alkyl, substituted or unsubstituted heteroalkyl, substituted or unsubstituted aryl, substituted or unsubstituted heteroaryl, substituted or unsubstituted heterocycloalkyl moieties.

The present invention also provides a novel ganglioside compound as described above, with the proviso that when X is NHR⁴, in which R⁴ is selected from H and —C(C═O)R⁵, in which R⁵ is substituted or unsubstituted alkyl, Y is OH; and Z is O, R⁵ is other than a substituted or unsubstituted alkyl group.

The present invention also provides a novel ganglioside compound in which the saccharide component is

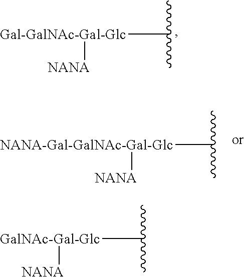

and such saccharide moieties may or may not be deacetylated. In certain embodiments, the saccharide component is Galβ(1,3)GalNAcβ(1,4)[NANAα(2,3)]Galβ(1,4)Glc−.

In some embodiments, the compound is of formulae VI, VII, VIII, or IX:

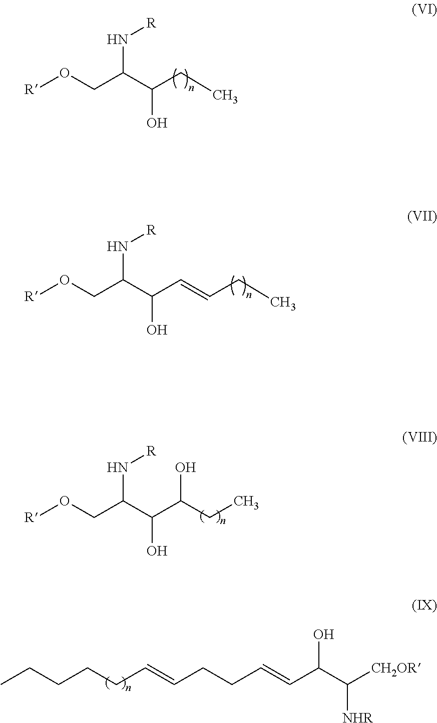

wherein n is an integer from 0 to 40, NHR is NH₂ or an amide of a long chain or short chain saturated, unsaturated, or polyunsaturated (i.e., having at least two double bonds in the fatty acid chain) fatty acid having from 1 to 40 carbons in the chain (e.g., formate, acetate, etc with and without unsaturation); and R′ is the saccharide moiety. In some embodiments, n is an integer selected from the group consisting of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40. More particularly, in some embodiments, n is 5, 12, 13, 14, 15, 16, 18 or 20. In some embodiments, the fatty acid is unsubstituted or alpha-hydroxylated. In some embodiments, the fatty acid is dichloroacetic acid. In other embodiments the fatty acid is substituted. In other embodiments still, the R fatty acid member has from 14 to 24 carbon atoms in the fatty acid chain. In some embodiments, the long chain fatty acid is an unsubstituted or an alpha-hydroxylated C16, C18, C20, C22, or C24 fatty acid. In some embodiments, the fatty acid is an unsubstituted omega-3 or omega-6 or omega 9 fatty acid which may be optionally substituted with an alpha-hydroxyl group. In some further embodiments of such, the fatty acid is a C16, C18, or C18 fatty acid. In some embodiments, the compound comprises a 4,8-sphingadiene backbone. In some further embodiments of any of the above, the fatty acid is a polyunsaturated fatty acid. In some embodiments of the above, the fatty acid is an α-hydroxy fatty acid (e.g., a substituted or unsubstituted α-hydroxy palmitic acid or stearic acid) joined at the amide bond to a sphingoid base. In some embodiments of the above, the long chain fatty acid is α-hydroxy palmitic acid. In other embodiments of any of the above, R′ is the saccharide moiety of a ganglioside selected from GM1b, GM3, GM2, GM1a, GD1a, GD1b. In an embodiment, the saccharide moiety is a mono-, di-, tri-, tetra-, penta-, hexa-, or hepta-saccharide. In exemplary embodiments, the compound of formula (I) to (IX) has the stereoisomerism of a corresponding naturally occurring ganglioside. In some embodiments, wherein the ganglioside has one or more double bond, the double-bond may be independently cis or trans, or a mixture thereof. In some embodiments, the double bonds are in the trans configuration. In yet other embodiments, the compounds for use according to the invention, including the compounds of formulae (I) to (IX), comprise an omega-3 fatty acid (e.g., DHA (docosahexaenoic acid), EPA (eicosapentaenoic acid) or an omega-6 fatty acid (e.g., GLA (gamma linolenic acid)) as the long chain polyunsaturated fatty acid. In a further set of embodiments of such compounds, n is 14. In a still further embodiment of such, the saccharide moiety is the oligosaccharide moiety of GM3, GM2, GM1a, GD1a.

In another aspect of the invention there is provided a method of treating a disease or disorder mediated by a GM1 deficiency in a mammal comprising administering to the mammal an effective amount of a compound of the formula (I)-(V) wherein

### X is H, D, —OR₃, —NR₃R₄, —SR₃ or —CHR₃R₄;

Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;

### Z is O, S or NR₆;

R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and

### M is O, S or NR₆.

In another aspect of the invention, there is provided a method of increasing cognitive or motor function or decreasing cognitive or motor function decline a disease or disorder mediated by a GM1 deficiency in a mammal comprising administering to the mammal an effective amount of a compound of the formula (I)-(V) wherein:

### X is H, D, —OR₃, —NR₃R₄, —SR₃ or —CHR₃R₄;

Y is H, —OR⁷, SR⁷, —NR⁷R⁸, substituted or unsubstituted alkyl, substituted or unsubstituted hetero4444akly, substituted or unsubstituted aryl, substituted or unsubstituted heteroaryl, or substituted or unsubstituted heterocycloalkyl;

### Z is O, S or NR₆;

R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and

### M is O, S or NR₆.

In another aspect of the invention there is provided a method of preventing neuronal cell death comprising contacting said neuronal cell that is deficient in GM 1 with an effective amount of a compound of the formula (I)-(V) wherein:

### X is H, D, —OR₃, —NR₃R₄, —SR₃ or —CHR₃R₄;

Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;

### Z is O, S or NR₆;

R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and

### M is O, S or NR₆.

The invention further provides pharmaceutical compositions including at least one compound of the invention and a pharmaceutically acceptable carrier.

The present invention further provides a method for the prevention and/or treatment and/or cure of Parkinson's disease in an animal or human including the step of administering to a patient in need thereof a therapeutically effective amount of at least one compound or pharmaceutical composition of the invention. Such patients in need of a compound of the present invention may suffer from motor dysfunctions that include one or more of tremor, rigidity, speech impairment, loss of balance, muscle fatigue, muscle pain, dizziness, impaired movement, bradykinesia, impaired dexterity, impaired swallowing, impaired hand writing, twitching, dyskinesia, respiratory distress, impaired breathing, postural instability, postural disturbances (e.g. festination, forward-flexed posture), vision dysfunction, vision loss, incontinence, and blurred vision. Such patients in need of a compound of the present invention may also suffer from cognitive dysfunction or impairment that include one or more of memory disturbances, learning problems, executive dysfunction (e.g. problem solving), attention difficulties, slow thinking, language dysfunction, dementia, visual-spatial disturbances, mood difficulties (e.g. depression, anxiety, apathy), impulse control dysfunction (e.g. obsessive behaviors), insomnia, and psychosis (e.g. hallucinations, delusions). In an embodiment, administration of the compound provides an improvement in one or more of the motor and/or cognitive dysfunctions. In still other embodiments, patient improvement is measured by methods known in the art (e.g. UPDRS, MDRS, MMSE, CGI, ADAS-cog, ADCS-ADL, NPI-10 and ADCS-CGIC) and the response is statistically significant (e.g. student T-test or the like).

In another embodiment, the pharmaceutical compositions are in unit dose format and each unit dose provides a therapeutically effective amount of one or more compounds for use according to the invention. The compositions, in some embodiments, are formulated for oral, intraperitoneal, intracerebroventricular, intrathecal, subcutaneous, or intravenous administration. In another embodiment, the formulated compound is administered by oral, intraperitoneal, intracerebroventricular, intrathecal, subcutaneous, or intravenous route. In some embodiments, the compound is administered by continuous infusion. In another embodiment, the compound is administered as single doses. In still other embodiments, the compound is administered using a pump. In some embodiments, the pump is implanted internally on the subject. In other embodiments, the pump is utilized externally on the subject.

In a particular embodiment, the pump is a Medtronic SynchroMed II pump.

In some embodiments, the above compositions can comprise a plurality of ganglioside analogs wherein each has a saccharide moiety of a different ganglioside of the same mammalian synthetic pathway. In some such embodiments, the administered gangliosides consist of at least two or three such gangliosides. In still other embodiments, the administered gangliosides consist of at least 50%, 70%, 80%, 90%, 95%, or 98% of a single ganglioside having a saccharide or R′ moiety of a naturally occurring mammalian ganglioside.

In another aspect, the invention provides methods of treating Parkinson's disease associated with an increased aggregation of α-synuclein protein by administering to a subject having the disease or condition a therapeutically effective amount of a compound of Formula I, II, III, IV, or V. In this aspect, the saccharide moiety may be, but is not limited to, the oligosaccharide moiety or glycoform of GM3, GM2, GM1a, GD1a and GD1b. In an embodiment, the saccharide moiety is oligosaccharide moiety or glycoform of GM3, GM2, GM1a, GD1a or GD1b. In a further embodiment, the oligosaccharide moiety of the compound is that of GM1a. In some embodiments, the compound reduces or eliminates the α-synuclein aggregates. In still another embodiment, the reduction in α-synuclein aggregates increases neuron numbers. In an embodiment, the effected neurons are dopaminergic neurons, such as neurons that contain tyrosine hydroxylase, and/or cortical neurons.

In another embodiment, administration of the compound restores the biological functions of the neurons such as neurotransmitter production and response. In a particular embodiment, the neurons produce greater amounts of dopamine. In still another embodiment, the treatment restores dopamine transporter levels. In still another embodiment, the improvements are measured by methods known in the art (e.g. MRI, PET, SPECT, DaTscan and CAT) and the response is statistically significant (e.g. student T-test or the like).

In another aspect, the invention provides methods of treating Parkinson's disease associated with an increased aggregation of α-synuclein protein in a subject by administering to the subject one or more of the compounds of Formula I, II, III, IV or V in which the compound(s) has a saccharide moiety of the deficient ganglioside or of a ganglioside which is downstream of a deficient ganglioside in a subject with the disease or condition. In some embodiments, the ganglioside deficiency disease may be mediated or caused by the condition or disease to be treated or by a reduced level of activity or absence of any of the enzymes involved in mammalian ganglioside synthesis or anabolism (e.g., GM2 synthase, galactosyltransferases, ST3 Gal5).

In one embodiment of the above, the deficient ganglioside is GM1b, GD1c, Gd1a, GM3, GM2, GM1a, GD1a, GT1a, GT1α, GD3, GD2, GD1b, GT1b, GQ1b, GQ1bα, GT3, GT2, GT1c, GQ1c, GP1c, or GP1cα and the compound for use according to the invention to be administered has an oligosaccharide moiety of the deficient ganglioside or a ganglioside downstream of the deficient ganglioside in the corresponding mammalian biosynthetic pathway. Subsequent metabolism of the administered compound can generate other glycoforms which have oligosaccharide moieties corresponding to the first deficient ganglioside in the biosynthetic pathway as well as other deficient gangliosides which are downstream of the first deficient ganglioside in the biosynthetic pathway.

In another aspect, the invention provides a method of treating a subject for Parkinson's disease associated with reduced activity of the GDNF receptor, RET, by administering to the subject a compound of Formula I, II, III, IV, or V. In some further embodiments, administration of the compound increases RET activity. In still other embodiments, the increase in RET signaling improves the motor and/or cognitive function of the treated subject. In another embodiment, the increased RET activity increases cell amounts. In an embodiment, the neurons are dopaminergic neurons or cortical neurons. In another embodiment, administration of the compound restores the biological function of the neurons.

In a particular embodiment, the treated neurons produce greater amounts of dopamine or other neurotransmitters (e.g. acetylcholine, glutamate, seratonin). In still other embodiments, the cell numbers and/or neurotransmitter amounts are measured by methods known in the art (e.g. MRI, PET, SPECT) and the effects are statistically significant (e.g. student T-test or the like). In still another embodiment, the treatment restores dopamine transporter (e.g. DAT, VMAT) levels. In other embodiments, the compound when used in combination with GDNF treatment enhances the motor and/or cognitive functions of the subject. In still other embodiments, the enhanced effect is synergistic.

### BRIEF DESCRIPTION OF THE DRAWINGS

FIG. 1. GM1 deficiency in the substantia nigra pars compacta (SNpc) neurons of PD patients correlates with a decline in TH+ neurons and an increase in α-synuclein (αSyn) aggregates. IHC of GM1 and TH+ cells (a) and GM1 plus αSyn (b) in paraffin sections from SNpc region of one PD patient and one non-PD age matched control. Upper panels indicate phase contrast microscopy. (c) Quantification for 11 sporadic PD patients and 11 non-PD age matched controls. Eight sections of each patient were counted. Both groups: 66-92 years of age (males and females). Neuropathological stage of PD ranged from 4/6 to 6/6. Total dopaminergic (DA) neurons are the sum of tyrosine hydroxylase positive (TH+) neurons and alpha-synuclein (αSyn+) neurons. GM1 staining with cholera toxin B linked to FITC is seen in the plasma membrane (arrows) and nuclear envelope (arrow heads). DA neurons with GM1 include those detected with GM1 at one or both locations.

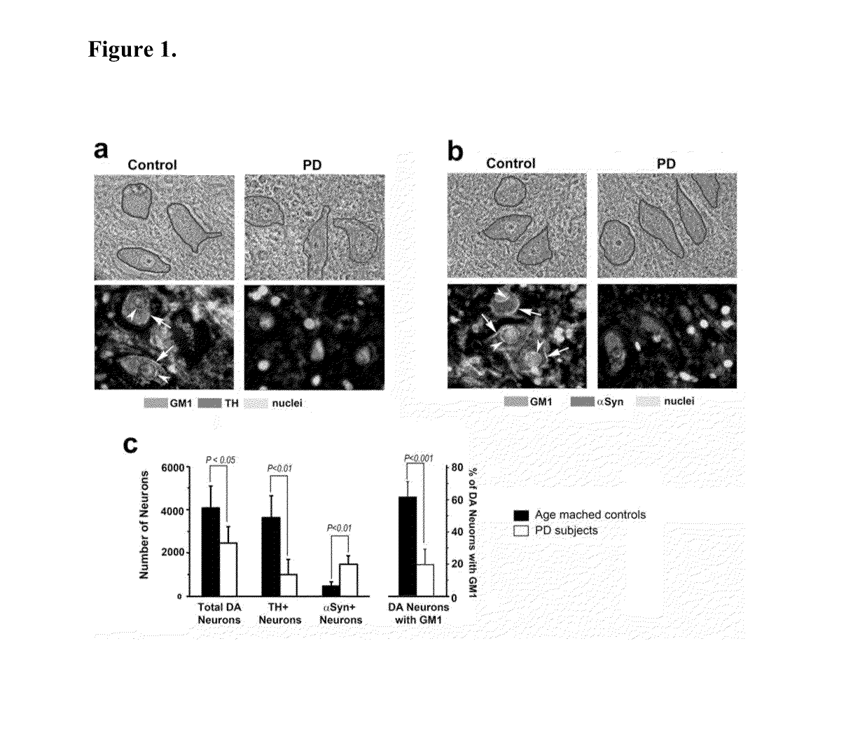

FIG. 2. Heterozygous (HT) mice partially deficient in GM1 develop PD neuron phenotype in SNpc with aging. Age-related changes in SNpc include: (a) GM1 decrease, (b) % of TH+ cells with GM1 decrease, (c) % of TH+ cells with αSyn aggregates increase, (d) dopamine level decrease, (e) TH+ neuron number decrease. Mice were ˜200 days of age (DOA).

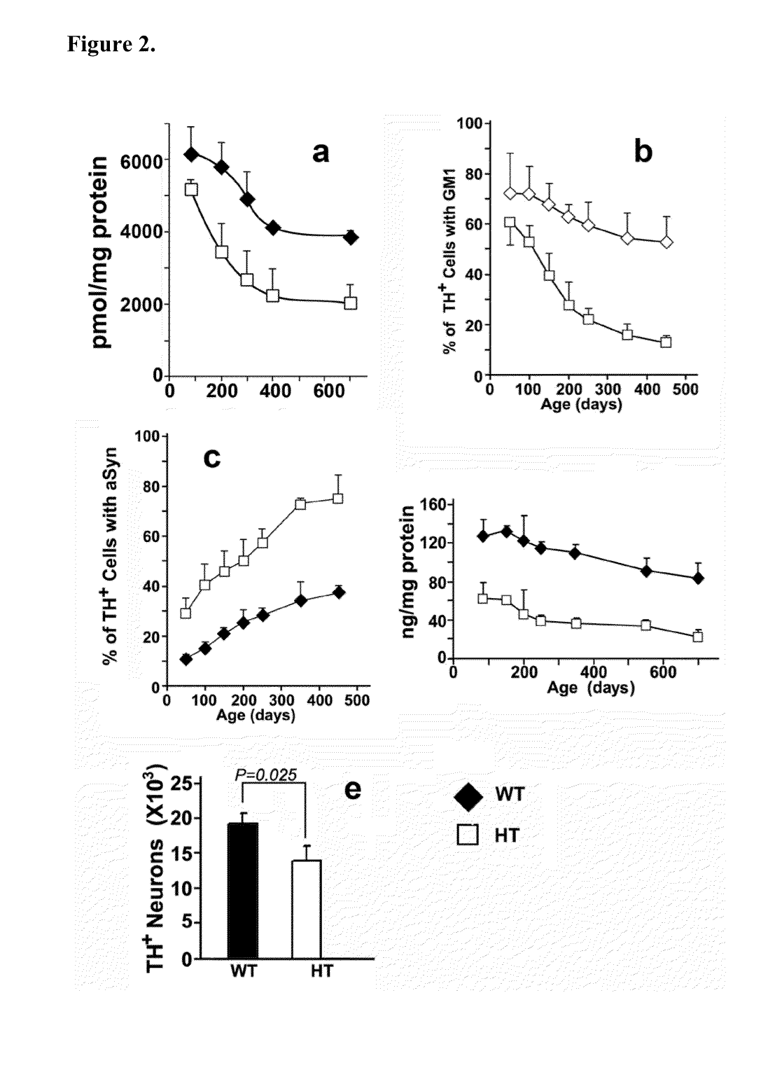

FIG. 3. Reversal of PD neuropathology by GM1 replacement. HT mice (200 days old) were treated with saline or compound XVIII (2.5 mg/Kg; 3×/wk, ip) over 5 weeks. Changes observed in TH+ cells: (a) neuron number increase; (b) αSyn aggregates decrease; (c) DA levels increase.

FIG. 4. L-DOPA alleviates motor deficits in GM1 Deficient Mice (HT). Grip duration (a) and irritant removal (b) were tested in mice at 200 DOA before and after IP injection of L-DOPA.

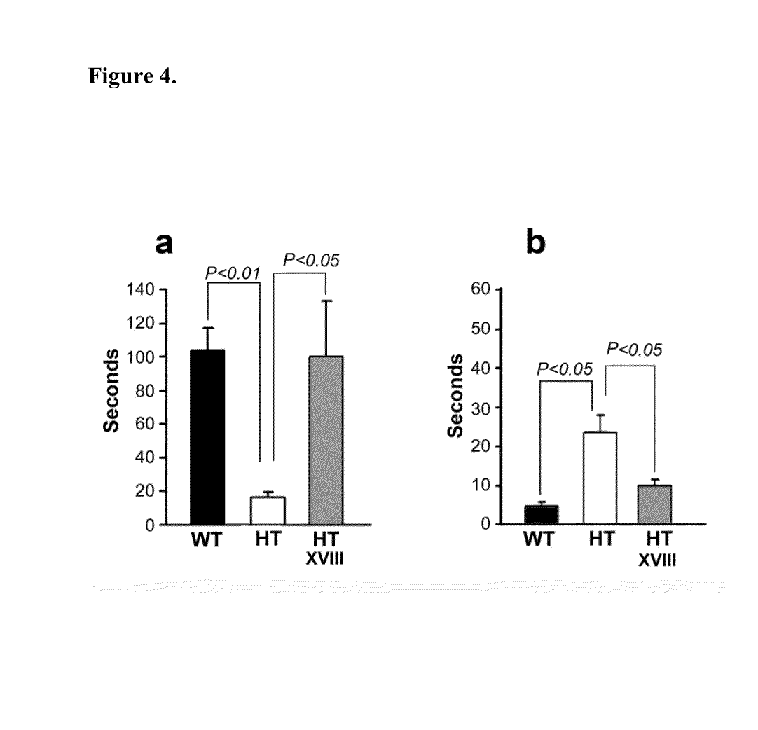

FIG. 5. GM1 replacement improves motor function in HT GM1 deficient mice. Mice (200 DOA) were treated IP with saline or compound XVIII (2.5 mg/Kg; 3×/wk) over 5 weeks. (a) grip duration; (b) irritant removal; (c) pole climbing.

FIG. 6. GM1 replacement improves cognitive function in HT GM1 deficient mice. T-maze test: 4 wks dosing with GM1 analog XVIII; 4 mice/group; age as indicated. The rate of alternation is a statistical plot as 0.5 (pure chance) to 1.0 (perfect score). Saline was administered as control.

FIG. 7. GM1 functional requirements. GM1 is required in cellular function for: 1. Neurotrophin signaling; 2.GDNF signaling; 3.Calcium homeostasis; and, 4.αSyn aggregate dissociation.

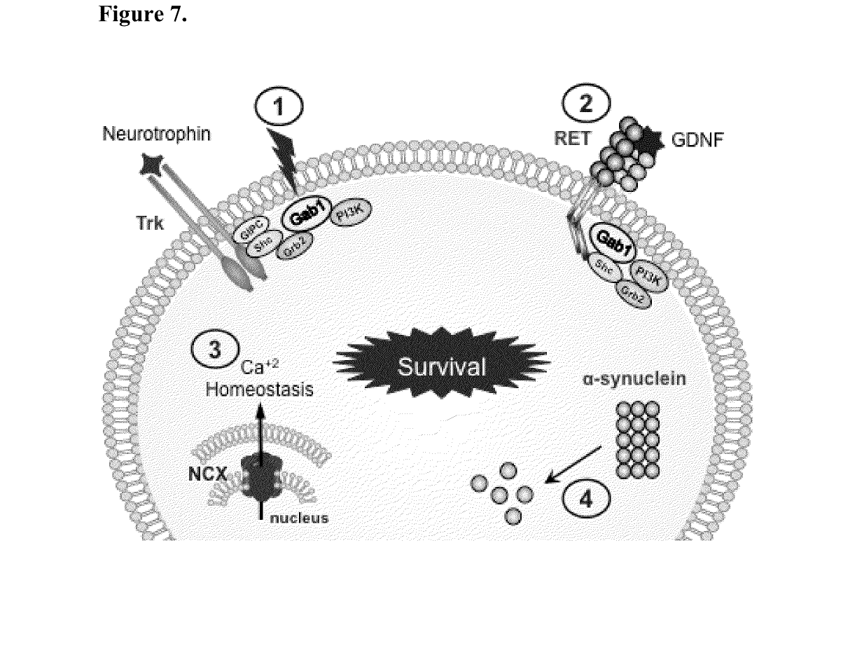

FIG. 8. GM1 replacement restores normal RET phosphorylation (RET-p) from subnormal state in GM1 deficient mice. IHC study with anti-RET-P and anti-TH revealed marked deficiency of RET-p in HT mice (low GM1) and in KO mice (no GM1). Treatment with GM1 analog XVIII significantly elevated RET-p in both genotypes.

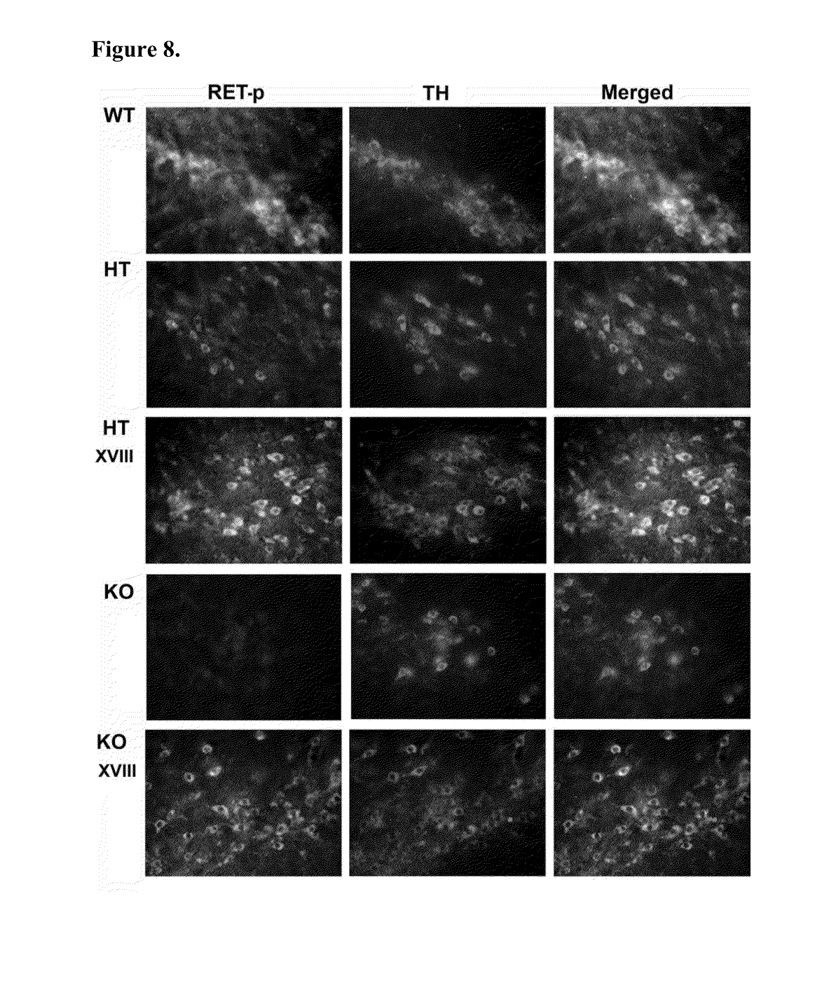

FIG. 9. Spontaneous formation of alpha-synuclein aggregates in cells deficient in GM1. NG-CR72 cells lacking GM1 were induced to differentiated with db-cAMP/KCl and showed expression of TH; staining with FITC-anti-αSyn antibody revealed abundant αSyn-aggregation that was suppressed with XVIII at 1 μM.

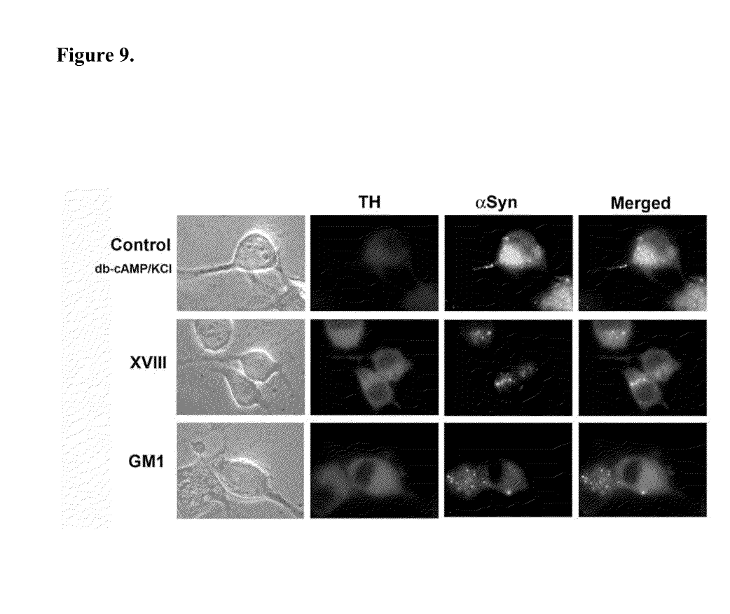

FIG. 10. GM1 analogs increase dopamine and TH+ cell numbers in the SNpc of MPTP treated mice. All agents administered once daily (I.P. or P.O.) for 3 weeks following MPTP treatment. (a) GM1 (1; 30 mg/Kg; IP), analog (3, 4 and 6; 0.3 mg/Kg; IP). (b) LIGA20 (2; 30 mg/Kg), analogs (2, 3 and 6; 30 mg/Kg; IP). (c) analogs (4 and 6; 30 mg/Kg; PO). (d) TH+IHC of the SNpc after treatment with saline or analog (6) (30 mg/Kg; PO) C=saline control. I and II are two separate experiments. Compound 2 is (XXIX/XXX), compound 3 is (XXVII/XXVIII), compound 4 is (XXXI/XXXII), compound 6 is (XXXVIII/XXXVII). LIGA20 is (XVIII).

FIG. 11. GM1 replacement therapy improves cortical and dopaminergic neuron survival. Compound 3 is (XXVII/XXVIII), compound 4 is (XXXI/XXXII), compound 5 is (XXXIII/XXXIV) and compound 6 is (XXXVIII/XXXVII). VMC are ventral mesencephalic neurons (dopaminergic neurons).

FIG. 12. GM1 replacement therapy reduces cell death and increases neurite outgrowth. NG108-15 neuroblastoma cells were differentiated for 7 days with db-cAMP/KCl, then treated 2 days with MPP and indicated compound for 2 days. (A) Relative cell number was significantly improved with LIGA-20 and compound 6 at 1 μM, equivalent to GM1 at 100 μM. (B) % of cells with neurites was similarly improved under those conditions. LIGA-20 is GM1 analog XVIII and compound 6 is analog XXXVII/XXXVIII.

### DEFINITIONS

In accordance with the invention and as used herein, the following terms are defined with the following meanings, unless explicitly stated otherwise.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented.

Atypical Parkinsonian diseases or syndromes refer to a group of disorders whose clinical features overlap those of idiopathic Parkinson's disease. The four major Parkinsonian syndromes embrace three important neurodegenerations, multiple system atrophy, progressive supranuclear palsy, and corticobasal degeneration, and a lacunar cerebrovascular disorder, vascular parkinsonism (see, Gilman S. Clin. Geriatr. Med 22:827-42 (2006). In an embodiment, the condition to be treated according to the invention is one of these four major syndromes.

The article “a” and “an” as used herein refers to one or to more than one (i.e. at least one) of the grammatical object of the article. By way of example, “an element” means one element or more than one element.

The term “sialic acid” refers to any member of a family of nine-carbon carboxylated sugars. Also included are sialic acid analogues that are derivatized with linkers, reactive functional groups, detectable labels, components for inclusion into lipid rafts and targeting moieties. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C.sub.1-C.sub.6 acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids: Chemistry, Metabolism and Function, R. Schauer, Ed. (Springer-Verlag, N.Y. (1992)). The synthesis and use of sialic acid compounds in a sialylation procedure is disclosed in international application WO 92/16640, published Oct. 1, 1992.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

Oligosaccharides described herein are generally described with the name or abbreviation for the non-reducing saccharide (Le., Gal), followed by the configuration of the glycosidic bond (.alpha. or .beta.), the ring bond (1 or 2), the ring position of the reducing saccharide involved in the bond (2, 3, 4, 6 or 8), and then the name or abbreviation of the reducing saccharide (i.e., GlcNAc). In an embodiment, each saccharide is a pyranose. For a review of standard glycobiology nomenclature see, Essentials of Glycobiology Varki et al. eds. CSHL Press (1999).

“Glycosphingolipid analogue” and “glycosphingolipid” are used herein to refer to the compounds of the invention. The terms are used to refer to glycosphingolipid structures in which the saccharyl moiety, the base (e.g., sphingoid-like backbone), or the fatty acid-derived hydrocarbon is of a structure other than that found in naturally occurring glycosphingolipids.

As used herein, the term “saccharide” may be used interchangeably with the term “carbohydrate” and refers to single simple sugar moieties or monosaccharides as well as combinations of two or more single sugar moieties or monosaccharides covalently linked to form disaccharides, oligosaccharides, and polysaccharides. The term “saccharide” also includes N-acetylated and N-deacylated derivatives of such monosaccharides, disaccharides, oligosaccharides, and polysaccharides. Saccharides for use in the invention may be linear or branched. Examples of suitable monosaccharides include, but are not limited to, known aldoses and ketoses (i.e. aldehyde and ketone derivatives of straight-chain polyhydroxy alcohols containing at least three carbon atoms) including, for example, glyceraldehyde, erythrose, threose, ribose (Rib), arabinose (Ara), xylose (Xyl), lyxose (Lyx), allose, altrose, glucose (Glc), mannose (Man), gulose, idose, galactose (Gal), talose, dihydroxyacetone, erythrulose, ribulose, xylulose, psicose, fructose (Frc), sorbose, and tagatose. Other examples of suitable monosaccharides include, but are not limited to, fucose (Fuc), N-acetylneuraminic acid (also called sialic acid, NANA, or NAN (Sia)), N-acetylglucos amine (GlcNAc), and N-acetylgalactosamine (GalNAc). The cyclic hemiacetal and hemiketal forms of the monosaccharides are contemplated within the defined term.

As used herein, the term “disaccharide” refers to a saccharide composed of two monosaccharides linked together by a glycosidic bond. Examples of disaccharides include, but are not limited to, lactose (Lac) (glycosidic bond between Gal and Glc), sucrose (Suc) (glycosidic bond between Frc and Glc), and maltose (Mal), isomaltose and cellobiose (glycosidic bond between Glc and Glc).

The term “oligosaccharide” includes an oligosaccharide that has a reducing end and a non-reducing end, whether or not the saccharide at the reducing end is in fact a reducing sugar. In accordance with accepted nomenclature, an oligosaccharide is depicted herein with the non-reducing end on the left and the reducing end on the right. An oligosaccharide described herein may be described with the name or abbreviation for the non-reducing saccharide (e.g., Gal), followed by the configuration of the glycosidic bond (α or β), the ring bond, the ring position of the reducing saccharide involved in the bond, and then the name or abbreviation of the reducing saccharide (e.g., GlcNAc). The linkage between two sugars may be expressed, for example, as 2,3,2>3, 2-3, or (2,3).

The term “sphingoid,” as used herein, includes sphingosines, phytosphingosines, sphinganines, ceramides, and the like. Both naturally occurring and synthetically produced compounds are included.

The term “glycosphingolipid” is a carbohydrate-containing derivative of a sphingoid or ceramide. The carbohydrate residue is attached by a glycosidic linkage to 0-1 of the sphingoid.

The term “alkenyl” as used herein refers to a substituted or unsubstituted straight chain or branched chain unsaturated aliphatic radical that includes at least two carbons joined by a double bond. Examples of alkenyl groups include, but are not limited to, vinyl, 2-propenyl, 2-isopentenyl, 2-(butadienyl), 2,4-pentadienyl, 3-(1,4-pentadienyl), and the higher homologs and isomers.

The term “alkynyl” as used herein refers to a straight or branched chain aliphatic radical that includes at least two carbons joined by a triple bond. If no number of carbons is specified, “alkenyl” and “alkynyl” each refer to radicals having from 2-12 carbon atoms. Examples of alkynyl groups include, but are not limited to ethynyl, 1- and 3-propynyl, 3-butynyl, and the higher homologs and isomers.

The term “cycloalkyl” as used herein refers to a substituted or unsubstituted saturated aliphatic mono-, bi-, or tricyclic saturated aliphatic ring system. Examples include, but are not limited to, cyclopropyl, cyclobutyl, cyclopentyl, cyclohexyl, adamantyl, cyclooctyl, [3.3.0]bicyclooctane, [4.3.0]bicyclononane, [4.4.0]bicyclodecane (decalin), and [2.2.2]bicyclooctane.

The term “aromatic” is intended to mean stable substituted or unsubstituted mono-, bi-, or tri-, polycyclic ring structures having only carbon atoms as ring atoms including, but not limited to, a stable monocyclic ring which is aromatic having six ring atoms; a stable bicyclic structure having a total of from 7 to 12 carbon atoms in the two rings of which at least one of the rings is aromatic; and a stable tricyclic ring structure having a total of 10 to 16 atoms in the three rings wherein the tricyclic ring structure of which at least one of the ring is aromatic. Any non-aromatic rings present in the monocyclic, bicyclic, or polycyclic ring structure may independently be saturated, partially saturated or fully saturated. Examples of such “aromatic” groups include, but are not limited to, phenyl and naphthyl.

The term “arylalkyl” as used herein refers to one, two, or three substituted or unsubstituted aryl groups having the number of carbon atoms designated appended to an alkyl group having the number of carbon atoms designated. The direction of attachment of an arylalkyl group to the remainder of the molecule may be through either the aryl or alkyl portion of the group. Suitable arylalkyl groups include, but are not limited to, benzyl, piclyl, naphthylmethyl, penethyl, benzylhydryl, trityl, and the like, all of which may be optionally substituted.

As used herein the term “hereroaryl,” “heteroaromatic” or aromatic heterocyclic ring system” refers to a monocyclic, bicyclic or polycyclic, substituted or unsubstituted heterocyclic ring system containing at least one aromatic ring.

The term “substituted” as used herein means that a hydrogen atom has been replaced with another monovalent group (e.g. halo, haloalkyl, hydroxy, thiol, alkoxy, thiohaloalkyl, amino, and the like), or that two hydrogen atoms of the same atom have been replaced by a divalent group.

The terms “halo” or “halogen” as used herein refer to Cl, Br, F or I. The term “haloalkyl” and the like, refer to an alkyl group, as defined herein, wherein at least one hydrogen atom of the alkyl group is replaced by a Cl, Br, F or I. A mixture of different halo atoms may be used if more than one hydrogen atom is replaced. For example, a haloalkyl includes chloromethyl (—CH₂Cl) and trifluoromethyl (—CF₃) and the like.

The term “methylene” refers to —CH₂—.

The letter “D” in the definitions of groups, for example in R1-R5, means deuterium.

Where substituent groups are specified by their conventional chemical formulae, written from left to right, they equally encompass the chemically identical substituents which would result from writing the structure from right to left, e.g., —CH₂O— is intended to also recite —OCH₂—.

The term “alkyl,” by itself or as part of another substituent, means, unless otherwise stated, a straight or branched chain, or cyclic hydrocarbon radical, or combination thereof, which is saturated, and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like.

The term “alkylene” by itself or as part of another substituent means a divalent radical derived from an alkane, as exemplified, but not limited, by —CH₂CH₂CH₂CH₂—. Typically, an alkyl (or alkylene) group will have from 1 to 24 carbon atoms. In an embodiment, alkyl (or alkylene) has 10 or fewer carbon atoms. In an embodiment, alkyl (or alkylene) has 4 carbon atoms. In an embodiment, alkyl (or alkylene) has 3 carbon atoms. In an embodiment, alkyl (or alkylene) has 2 carbon atoms. In an embodiment, alkyl (or alkylene) has 1 carbon atom. A “lower alkyl” or “lower alkylene” is a shorter chain alkyl or alkylene group, generally having six or fewer carbon atoms.

The terms “alkyl,” “alkenyl” and “alkynyl” are meant to include both substituted and unsubstituted forms of the indicated radical. The terms also include, but are not limited to, forms of the radicals having 3 or fewer or 6 or fewer carbon atoms. Particular substituents for each type of radical are provided below.

The term “alkoxy,” alkylamino” and alkylthio” (or thioalkoxy) are used in their conventional sense, and refer to those alky groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen and sulfur atoms may optionally be oxidized and the nitrogen heteroatom may optionally be quaternized. The heteroatom(s) O, N an S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, Si(CH₃)₃, —CH₂—CH═N—OCH₃, and —CH═CH—N(CH₃)—CH₃. Up to two heteroatoms may be consecutive, such as for example, —CH₂—NH—OCH₃ and —CH₂—O—Si(CH₃)₃. Similarly the term “heteroalkylene” by itself or as part of another substituent means a divalent radical derived from heteroalkyl, as exemplified, but limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heterolkylene linking groups, no orientation of the linking group is implied by the direction in which the formula of the linking group is written. For example, the formula —C(O)₂R′— represents both —C(O)₂R′— and —R′C(O)₂—.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) is one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)—NR″″, —NR—C(NR═R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. In an embodiment, R′, R″, R′″ and R″″ each independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they may be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

The term “cycloalkyl” and heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropridyl), 1-piperidinyl, 2-piperidinyl, 3-piperidinyl, 4-piperidinyl, 4-morpholinyl, 3-morpholinyl, tetrahydrofuran-2-yl, tetrahydrofuran-3-yl, tetrahydrothein-2yl, tetrahydrothein-3-yl, 1-piperazinyl, 2-piperazinyl, and the like.

Similar to the substituents described for the alkyl radical, substituents for the aryl and heteroaryl groups are varied and are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—CR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system. When a compound of the invention includes more than one R group, for example, each of the R groups is independently selected as are each R′, R″, R′″ and R″ groups when more than one of these groups is present.

The term “aryl” means, unless otherwise stated, a polyunsaturated, aromatic, hydrocarbon substituent comprising a single ring or multiple rings (for example, from 1 to 3 rings), which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized.

A heteroaryl group is attached to the remainder of the molecule through a carbon atom or a heteroatom. In an embodiment, the heteroaryl group is attached to the remainder of the molecule through a heteroatom. In an embodiment, such heteroatom is N. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

In an embodiment, substituents for the alkyl, alkenyl, alkynyl radicals are one or more of a variety of groups selected from, but not limited to: —OR^{a}, ═O, ═NR^{a}, ═N—OR^{a}, —NR^{a}R^{b}—SR^{a}, -halogen, —OC(O)R^{a}, —C(O)R^{a}, —CO₂R^{a}, —CONR^{a}R^{b}, —OC(O)NR^{a}R^{b}, —NR^{b}C(O)R^{a}, —NR^{c}—C(O)NR^{b}R^{a}, —NR^{b}C(O)₂R^{a}, —NR^{e}—C(NR^{a}R^{b}R^{c})═NR^{d}, —NR^{d}—C(NR^{a}R^{b})═NR^{c}, —S(O)R^{a}, —S(O)₂R^{a}, —S(O)₂NR^{a}R^{b}, —NR^{b}SO₂R^{a}, —CN and NO₂ in a number ranging from zero to (2m′+1), where m′ is the total number of carbon atoms in such radical. In an embodiment, R^{a}, R^{b}, R^{c}, R^{d} and R^{e} each independently refer to hydrogen or unsubstituted alkyl. When a compound of the invention includes more than one of any R^{a}, R^{b}, R^{c}, R^{d} or R^{e} group, each of those groups are independently selected as well. For example, if there are two or more R^{a} groups in a formula, each of those are independently selected. When R^{a} and R^{b} are attached to the same nitrogen atom, they can optionally be combined with the nitrogen atom to form a 5-, 6-, or 7-membered ring. For example, —NR^{a}R^{b} is meant to include, but not be limited to, 1-pyrrolidinyl and 4-morpholinyl. From the above discussion of substituents, one of skill in the art will understand that the term “alkyl” is meant to include, but not be limited to, groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Particular substituents are lower alkyl, lower alkoxy, hydroxy, and halo. The term “lower” indicates C₁ to C₆ carbons in the chain. In some embodiments, the total number of substituents for the alkyl, alkenyl, or alkynyl radical are independently in a number which is 1, 2, 3 or 4. In a further embodiment, these substituents are independently selected from the group consisting of lower alkyl, lower alkoxy, hydroxy, or halo.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts may be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts may be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 66:1-19 (1977)). Certain specific compounds of the present invention contain both basic and acidic functionalities that allow the compounds to be converted into either base or acid addition salts. In an embodiment, the neutral forms of the compounds are regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs may be converted to the compounds of the present invention by chemical or biochemical methods in an ex vivo environment. For example, prodrugs may be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

“Pharmaceutically acceptable acid addition salt” as used herein refers to salts retaining the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicyclic acid and the like.

“Pharmaceutically acceptable base addition salts” as used herein refers to those salts derived from inorganic bases such as sodium, potassium, lithium, ammonium, calcium, magnesium, iron, zinc, copper, manganese, aluminum salts and the like. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like.

The term “biological property” as used herein means an in vivo activity that is directly or indirectly performed by a compound or pharmaceutical composition of the invention that is often shown by in vitro assays. In the present invention, the biological property is neuroprotection, including the prophylaxis, treatment and/or cure of Parkinson's disease and the associated motor and/or cognitive effects.

The term “isomer” as used herein refers to a compound having the same number and kind of atoms and hence the same molecular weight as another compound, but differing in respect to the arrangement or configuration of the atoms of the compound (e.g. cis and trans isomers), The term “isomer” also includes stereoisomers, diastereoisomers, enantiomers or mixtures thereof. In a particular embodiment, isomer is the D-isomer.

The term “substructure” as used herein refers to a portion of a chemical compound. For example, a single aromatic ring of a napthalene structure is herein referred to as a substructure of the entire napthalene molecule.

The term “hydrate” as used herein refers to the product of water with a compound of the invention such that the H—OH bond is not split. A compound of the invention may form more than one hydrate; however, the amount of water in a hydrate of the invention is such that the compound remains stable. In an embodiment, a hydrate of a compound of the invention contains about 0.1-10% water.

The term “deficiency state” refers to a harmful or large relative reduction in the levels of the referenced ganglioside or enzyme or enzyme activity in tissues or samples from a subject. Such deficiency states may generally exist, for instance, when the levels of a subject glycolipid are less than two-thirds, one-half, one-fourth or one-tenth of the levels for the comparable general population or a well-matched control group which is otherwise healthy or unaffected by the disease or condition being studied.

The term “fatty acid” refers to an aliphatic monocarboxylic acid which may be substituted or unsubstituted and saturated or unsaturated and have from 1 to 40 carbon atoms. In some embodiments, the fatty acid is from 2 to 40 carbon atoms in length, including the carboxyl moiety carbon. In further embodiments, the fatty acid is 14 to 24 carbon atoms in length and is saturated or unsaturated. In other embodiments, the fatty acid is unsubstituted and from 14 to 24 carbons in length and is saturated or unsaturated. If unsaturated, the double bond may be in the cis or trans conformation, or be a mixture thereof. In some embodiments, the fatty acid is unsaturated with at least one of the double bonds is in the trans configuration. Unsaturated fatty acids may be polyunsaturated, for instance, having from 1, 2, 3, or 4 double bonds. Exemplary fatty acids are stearic acid and oleic acid as well as the omega-3, omega-6, and omega 9 fatty acids of from 14 to 24 carbons in length. In some embodiments, the fatty acid is optionally substituted with an α-hydroxy or an α-alkoxy group or an acylated α-hydroxy group (acetyl α-hydroxy) which acylated compound may function as a prodrug of a compound of formula I, II, III, IV, or V. The aliphatic moiety may be linear or branched. In a particular embodiment, the aliphatic moiety is linear.

“Commercial scale” refers to gram scale production of a compound of formula I, II, III, IV, or V in a single reaction. In particular embodiments, commercial scale refers to production of greater than about 50, 75, 80, 90, 100, 125, 150, 175, or 200 grams of a compound of formula I, II, III, IV, or V. In some embodiments, the methods of use and manufacture of the pharmaceutical compositions involve the use of such compounds produced on a commercial scale.

The phrase “target volume of distribution at steady state” refers to a desired volume of tissue, such as brain tissue, receiving a solution containing a chemical moiety, where the solution has reached equilibrium inside the tissue, where the rate of delivery of the solution is equal to the rate of clearance of the solution. If a solution has reached the target volume of distribution at steady state, then the observed behavior of the solution remains constant. The target volume of distribution at steady state is not achieved until some time has elapsed after delivery of the solution is started or initiated.

A “therapeutic composition” or “treatment composition” means a substance that is intended to have a therapeutic effect such as pharmaceutical compositions, genetic materials, biologics, and other substances. Pharmaceutical compositions may be configured to function in an implanted environment with characteristics such as stability at body temperature to retain therapeutic qualities, concentration to reduce the frequency of replenishment, and the like.

Genetic materials include substances intended to have a direct or indirect genetic therapeutic effect such as genetic vectors, genetic regulator elements, genetic structural elements, DNA, iRNA and the like. Biologics include substances that are living matter or derived from living matter intended to have a therapeutic effect such as stem cells, platelets, hormones, biologically produced chemicals, and the like.

An “effective amount” means an amount of compound to be administered necessary to treat or prevent a disease or disorder mediated by GM 1 deficiency. Alternatively “effective amount” of the compound means the amount necessary to improve motor function in a mammal, or reduce a decline in motor function. Alternatively “effective amount” of the compound means the amount necessary to improve cognitive function in a mammal, or reduce a decline in cognitive function. Alternatively, “effective amount” is the amount of the compound required to prevent neuronal cell death. Alternatively, “effective amount” is the amount of the compound required to reduce the severity of a symptom of Parkinson's Disease.

A “therapeutically effective amount” is an amount of the therapeutic or treatment composition that provides a prophylactic or therapeutic benefit in the treatment, prevention, or management of a CNS disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective may be readily determined by ordinary medical practitioner, and may vary depending on factors known in the art, such as, e.g. the type of disease, the patient's history and age, the stage of disease, and the administration of other therapeutic agents.

Other objects, aspects and advantages of the present invention will be apparent from the detailed description below.

### DETAILED DESCRIPTION OF THE INVENTION

The present invention provides methods of treating Parkinson's disease by administering to a subject with such a condition, or suspected of having such a disease or condition, one or more compounds of Formula I, II, III, IV or V. The condition may be from a deficiency in the amounts or activity of an enzyme or substrate involved in the synthesis of a ganglioside deficient in a subject. The administered compounds of any formulae I to V can have saccharide or carbohydrate R′ moieties which correspond to the saccharide moiety of the first ganglioside which is deficient or of a ganglioside which is downstream of the first deficient ganglioside in the pertinent mammalian biosynthetic pathway of a ganglioside deficient in the subject. The administered compound may be converted in vivo to compound(s) having an oligosaccharide moiety of the deficient ganglioside or of other gangliosides downstream of the deficient ganglioside by the action of the endogenous catabolic and metabolic enzymes. For instance, where the defect is in GM3 synthase or GM3 levels, the subsequent metabolism of an administered compound of the invention having the oligosaccharide moiety of GM1 can provide corresponding compounds having the oligosaccharide moiety of GM2 and GD1a, thereby alleviating the deficiency state as to all three glycoforms.

The present invention further provides a method for the prevention and/or treatment and/or cure of Parkinson's disease in an animal or human including the step of administering to a patient in need thereof a therapeutically effective amount of at least one compound or pharmaceutical composition of the invention. Such patients in need of a compound of the present invention may suffer from motor dysfunctions that include one or more of tremor, rigidity, speech impairment, loss of balance, muscle fatigue, muscle pain, dizziness, impaired movement, bradykinesia, impaired dexterity, impaired swallowing, impaired hand writing, twitching, dyskinesia, respiratory distress, impaired breathing, postural instability, postural disturbances (e.g. festination, forward-flexed posture), vision dysfunction, vision loss, incontinence, and blurred vision. Such patients in need of a compound of the present invention may also suffer from cognitive dysfunction or impairment that include one or more of memory disturbances, learning problems, executive dysfunction (e.g. problem solving), attention difficulties, slow thinking, language dysfunction, dementia, visual-spatial disturbances, mood difficulties (e.g. depression, anxiety, apathy), impulse control dysfunction (e.g. obsessive behaviors), insomnia, and psychosis (e.g. hallucinations, delusions). In a particular embodiment, administration of the compound provides an improvement in one or more of the motor and/or cognitive dysfunctions. In still other embodiments, patient improvement is measured by methods known in the art (e.g. UPDRS, MDRS, MMSE, CGI, ADAS-cog, ADCS-ADL, NPI-10 and ADCS-CGIC) and the response is statistically significant (e.g. student T-test or the like).

In some embodiments, the compound of Formula I to V has a saccharide or R′ carbohydrate moiety selected from those set forth in the following table of gangliosides:

GalNAc4(Neu5Ac3)Gal4GlcCer GM2

Gal3GalNAc4(Neu5Ac3)Gal4GlcCer GM1a

Neu5Ac3Gal3GalNAc4Gal4GlcCer GM1b

Neu5Ac3Gal3GalNAc4(Neu5Ac3)Gal4GlcCer GD1a

Neu5Ac3Gal3(Neu5Ac6)GalNAc4Gal4GlcCer GD1α

Gal3GalNAc4(Neu5Ac8Neu5Ac3)Gal4GlcCer GD1b

The invention provides methods of treating Parkinson's disease; by administering glycolipids to a patient having the condition or signs or symptoms of the condition. In some embodiments, the glycolipid is a compound of Formula I, II, III, IV, or V. Several of these compounds have improved structures over lyso-GM1 and GM1(stearate) when injected or given orally. These structures exhibit improved biodistribution, (e.g., brain delivery), improved pharmacokinetics, and improved oral bioavailability.

Additionally the invention provides for the use of other glycolipids in treating Parkinson's disease including, for instance, the glycolipids of formula (I).

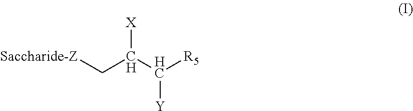

In formula (I):
the saccharide is as defined herein selected from the group consisting of a monosaccharide, a disaccharide, and oligosaccharide, a polysaccharide, an N-acetylated derivative thereof, and an N-deacylated derivative thereof:
Z is O, S, or —NR₁;
X is H, D, —OR₁, —NR₁R₂, —SR₁, or —CHR₁R₂;
R₁ and R₂ are independently H, D, —CH₂R₃, —C(=M)R₃, —C(=M)-p-R₃, —SO₂R₃, —SO₃, alkyl, cycloalkyl, aryl, arylalkyl, heteroaryl, heteroalkyl, or haloalkyl;
M is O, NR₄ or S;
R₄ is H, D, alkyl, cycloalkyl, aryl, arylalkyl, heteroaryl, heteroalkyl, or haloalkyl;
p is O, —NR₄ or S, where R₄ is set forth above;
R₃ is H, D, alkyl, cycloalkyl, arylalkyl, haloalkyl, aryl, heteroaryl, or heteroalkyl;
Y is H, D, —OR₁, —SR₁, —NR₁R₂, branched alkyl, cycloalkyl, aryl, arylalkyl, heteroaryl, heteroalkyl, or haloalkyl, here R₁ and R₂ is as set forth above; and
R₅ is H, D, alkyl, cycloalkyl, alkenyl, aryl, arylalkyl, heteroaryl, heteroalkyl, or haloalkyl;
and all pharmaceutically acceptable salts, isomers hydrates, prodrugs, and solvates thereof with the proviso that when Z is O, Y is OH and R₅ is alkenyl, X is not any of —NH₂, —NH(alkyl), —NHC(═O)alkenyl, —NHC(═O)fluoroalkyl, and —NHC(═O)alkyl.
   Compounds of the invention also include the compounds having the formula:

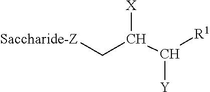

wherein X, Y, Z, R₁ and saccharide are as defined herein.

Compounds also include the neutral glycosphingolipids and neutral glycosyl sphingosines set forth in U.S. Patent Application Publication No. US 2005/0245735 which is incorporated by reference in its entirety.

Compounds of the invention also include sialylated oligosaccharide glycolipids disclosed in U.S. Patent Application Publication No. US 2005/0032742 which is incorporated herein by reference. In some embodiments, the oligosaccharide is a glycosylated ganglioside, ceramide, or sphingosine or an analogue of same. In some embodiments, the compound for use according to the invention is of the formula:

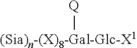

as defined therein.

The glycolipids for use in the methods of the invention are any of formula I to V herein and appendices A through C. When administered the glycolipids are administered in a “therapeutically effective amount” which refers to an amount of a therapeutic agent that is sufficient to benefit a subject suffering from a disease or condition. When a second agent is used with the compounds for use according to the invention the second compound is also used in a therapeutically effective amount. The amount(s) of one or both of agents used together may be adjusted downward when the two agents administered together act additively or synergistically.

The treatments according to the invention benefit a subject or patient by reducing the severity, intensity, duration, or frequency of the signs or symptoms of a condition or delaying onset. When a disease or condition is treated, the objective of the treatment may be to alleviate the condition generally or particularly with reference to any disclosed sign or symptom, or functional or structural impairment of the subject which is generally associated with the condition.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts may be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts may be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66, 1-19 (1977)). Certain specific compounds of the present invention contain both basic and acidic functionalities that allow the compounds to be converted into either base or acid addition salts.

In an embodiment, the neutral forms of the compounds are regenerated by contacting the salt with a base or acid and isolating the parent compound in the conventional manner. The parent form of the compound differs from the various salt forms in certain physical properties, such as solubility in polar solvents.

Certain compounds for use according to the present invention can exist in unsolvated forms as well as solvated forms, including hydrated forms. In general, the solvated forms are equivalent to unsolvated forms and are encompassed within the scope of the present invention. Certain compounds for use according to the present invention may exist in multiple crystalline or amorphous forms. In general, all physical forms are equivalent for the uses contemplated by the present invention and are intended to be within the scope of the present invention.

Certain compounds for use according to the present invention possess asymmetric carbon atoms (optical centers) or double bonds; the racemates, diastereomers, geometric isomers and individual isomers are encompassed within the scope of the present invention. Generally, exemplary compounds for use according to the invention possess the glycoform and/or stereoisomerism of the deficient ganglioside or one downstream thereof.

The compounds for use according to the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds for use according to the present invention, whether radioactive or not, are intended to be encompassed within the scope of the present invention.

In some embodiments, the compound of formula I to V is a glycolipid compound of a formula:

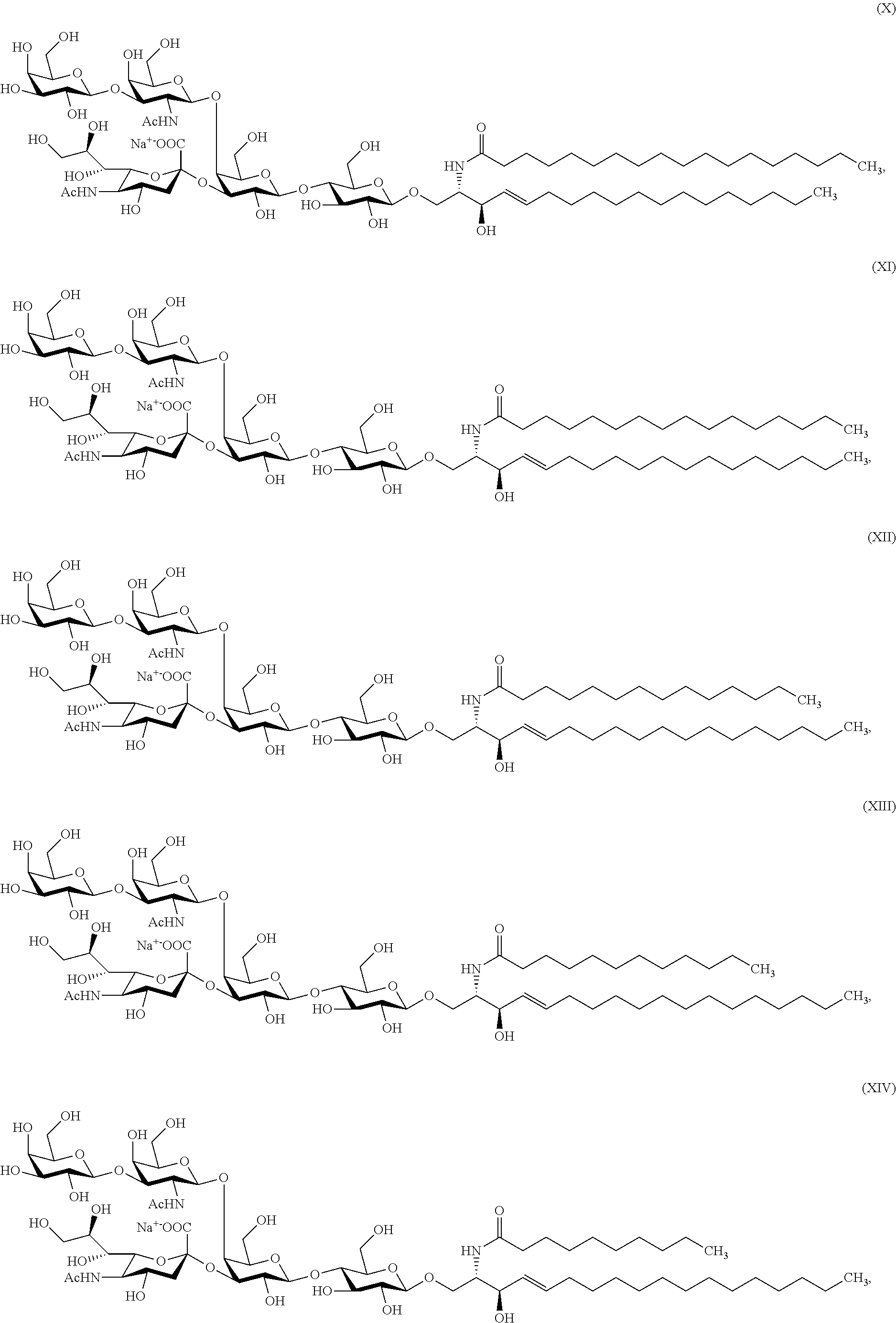

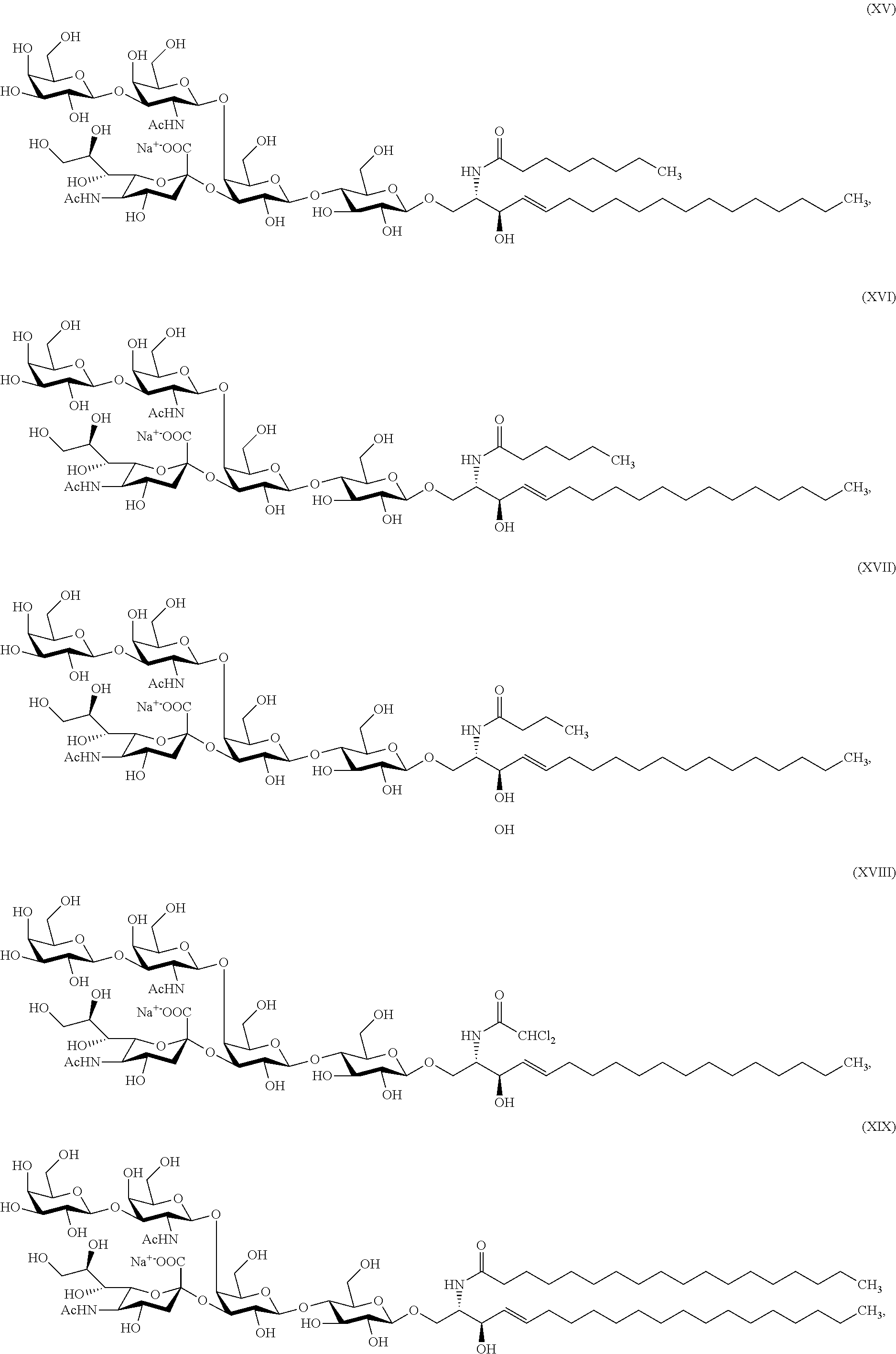

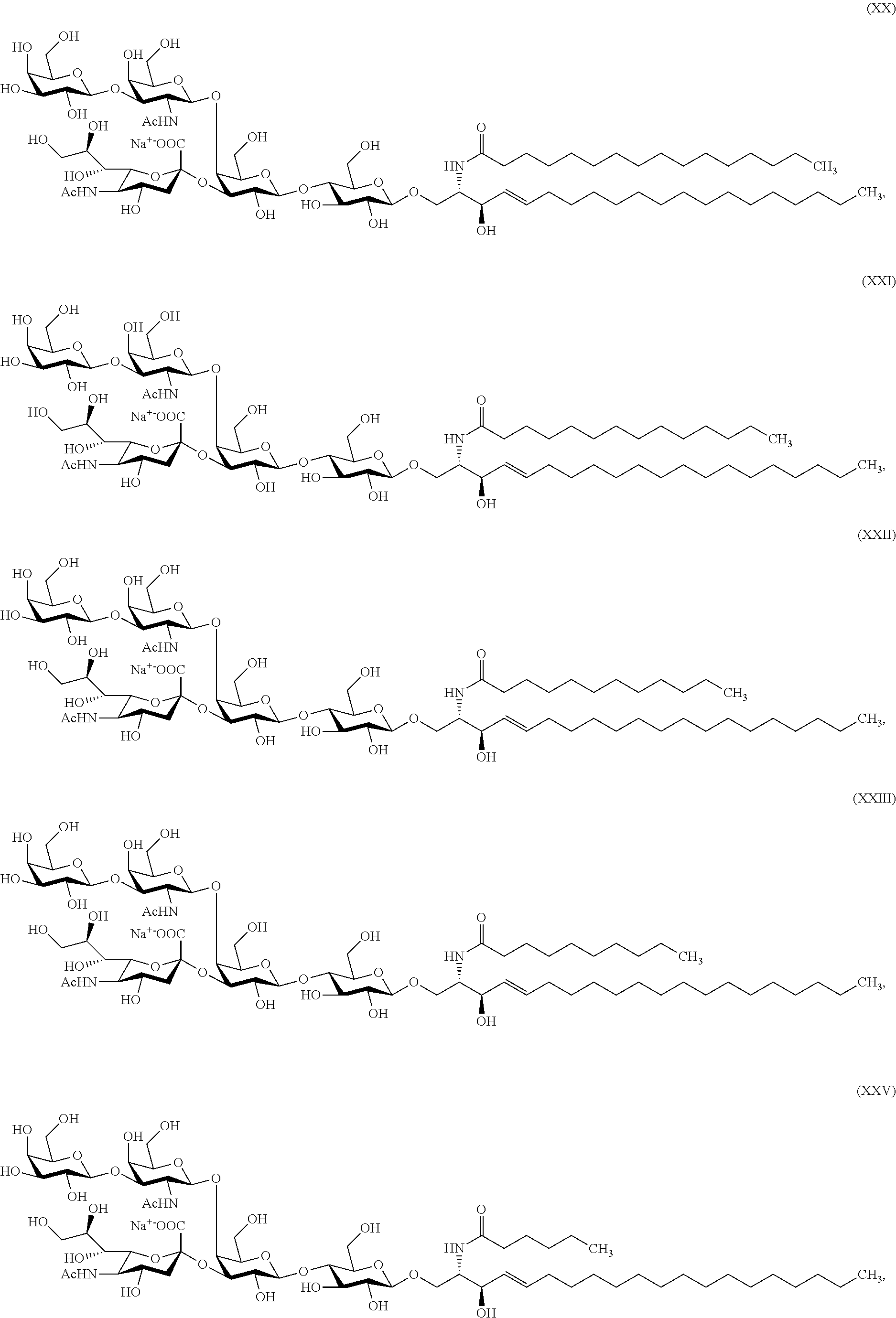

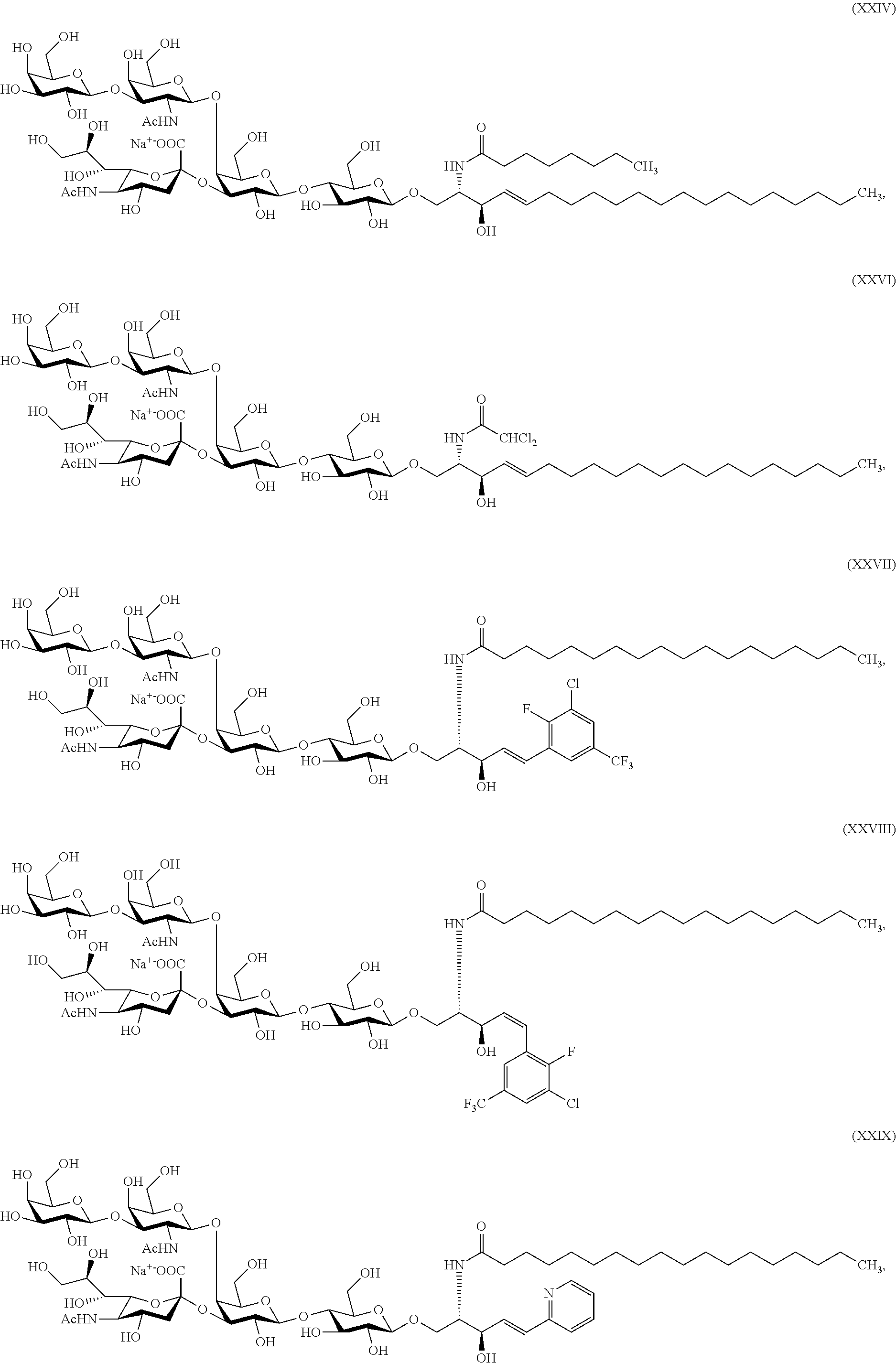

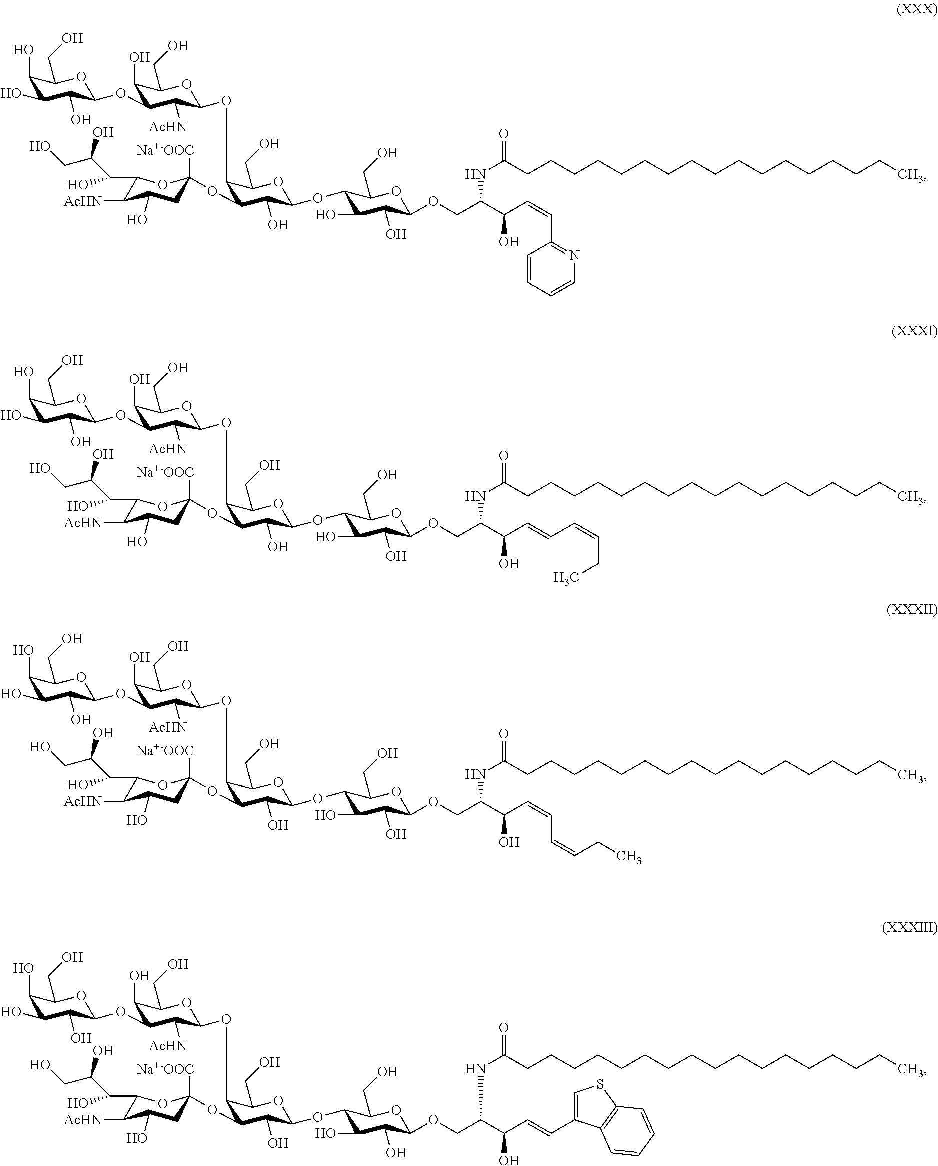

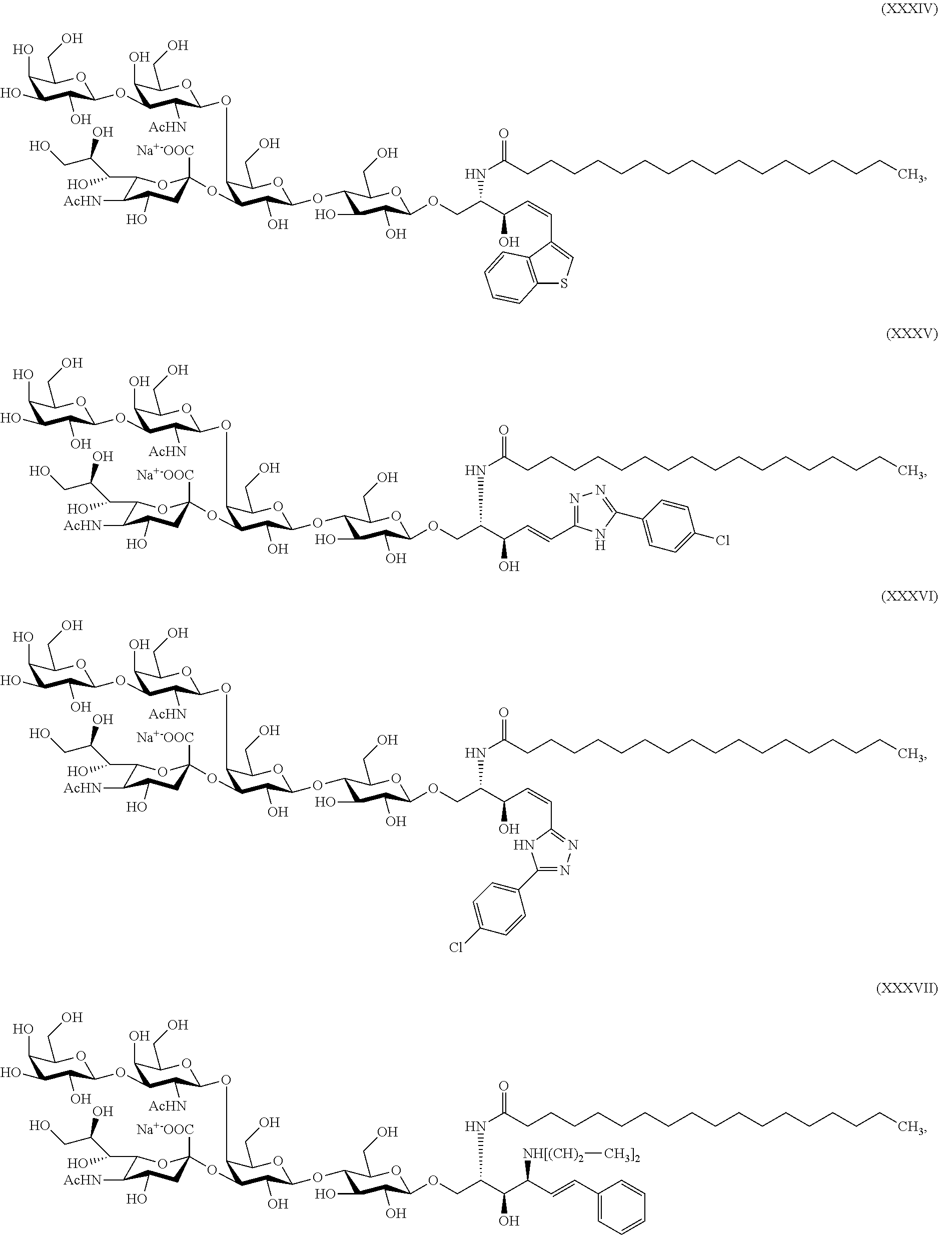

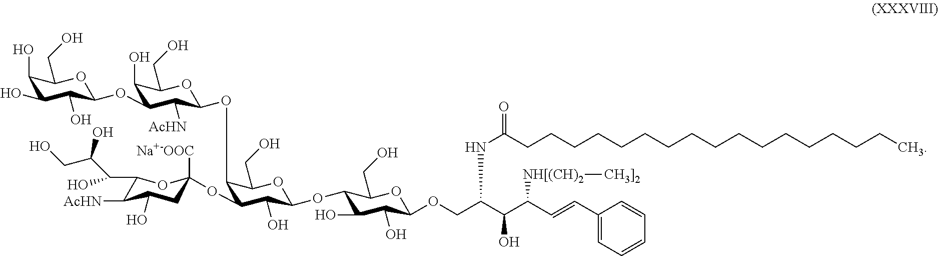

The compounds of formula I, II, III, IV, or V may be prepared by any method available to one of ordinary skill in the art. The saccharide moiety of the compounds for use according to the invention may be prepared by any means known in the art including those methods described in U.S. Pat. Nos. 5,922,577; 6,284,493; and 6,331,418, each of which is incorporated by reference herein in its entirety. Additional particularly suitable methods for making compounds for use according to the invention are described in U.S. Patent Applications Serial Nos. 60/576,316 filed Jun. 1, 2004; 60/626,791, filed Nov. 11, 2004; and 60/666,765, filed Mar. 29, 2005; which are assigned to the same assignee as the instant application; and in U.S. Pat. Nos. 6,440,703 and 6,030,815; and PCT International Patent Application Publications Nos. WO2004/080960, WO2003/016469, WO/2005/118798 and WO2004/080960. Each of these references are incorporated herein by reference particularly with respect to their teachings as to how to synthesize various glycolipids.

The compounds for use according to the invention may be prepared using, unless otherwise indicated, conventional methods and protocols in chemistry and enzymology known in the art. For example, compounds for use according to the invention may be prepared by chemical and enzymatic processes as outlined further below.

In an embodiment, the saccharide portion of the compounds for use according to the invention may be prepared enzymatically whereby a specific enzyme may be used to affect transfer of a monosaccharide from a donor molecule to an acceptor molecule, each as defined herein.

More specifically, disaccharides, oligosaccharides and polysaccharides, as found in the synthetic compounds for use according to the invention, may be prepared biosynthetically by the use of glycosyltransferases. Such glycosyltransferase reactions may be carried out in the presence of an organic solvent, such as, for example, methanol, ethanol, dimethylsulfoxide, isopropanol, tetrahydrofuran, chloroform, and the like, either singly or in combination. Alternatively, such glycosyltransferase reactions may be conducted in a biological medium in vitro, such as a biological buffer, a cell lysate, or on a chromatographic support, wherein the glycosyltransferase is immobilized on the chromatographic support and the other components of the reaction mixture are contacted with the glycosyltransferase by contacting the components with the chromatographic support in an aqueous medium.

Glycosyltransferase-mediated synthesis of saccharides may be conducted in vivo or in vitro. For example, whole-cell expression systems may be used for enzymatic synthesis, e.g., glycosyltransferase-mediated synthesis. Cell types useful for the expression of glycosyltransferases and production of saccharide structures include bacterial cells, yeast cells, and insect cells, as would be understood by one of skill in the art. The desired saccharide product may be isolated from the cell in which it was synthesized by lysis of the cell, or by isolation of cell culture medium when using a cell that secretes the saccharide product into the culture medium. The saccharide product may then be purified by means described elsewhere herein, or it may be used without further purification in a lysate or cell culture medium.

As understood by one of skill in the art, the enzyme used may vary depending upon the saccharide to be transferred to the donor. Examples of suitable enzymes include, but are not limited to, glycosyltransferases, trans-sialidases, and sialyltransferases. The choice of glycosyltransferase (s) used in a given synthesis method will depend upon the identity of the acceptor and donor molecules used as the starting material and the nature of the desired end product. The method can involve the use of more than one glycosyltransferase, where more than one saccharide is to be added. Multiple glycosyltransferase reactions may be carried out simultaneously, i.e., in the same reaction mixture at the same time, or sequentially.

Additional suitable synthetic approaches are disclosed in WO 2004/080960A2 which is incorporated by reference herein in its entirety.

If the acceptor is a ceramide, the enzymatic step is optionally preceded by hydrolysis of the fatty acid moiety from the ceramide. Methods of removing a fatty acid moiety from a glycosphingolipid are known to those of skill in the art. Standard carbohydrate and glycosphingolipid chemistry methodology may be employed, such as that described in, for example, Paulson et al., Carbohydrate Res. 137:39-62 (1985); Beith-Halahmi et al., Carbohydrate Res. 5:25-30 (1967); Alais and Veyrieries, Carbohydrate Res. 207:11-31; (1990); Grudler and Schmidt, Carbohydrate Res. 135:203-218 (1985); Ponpipom et al.; Tetrahedron Lett. 1717-1720 (1978); Murase et al., Carbohydrate Res. 188:71-80 (1989); Kameyama et al. Carbohydrate Res. 193:c1-c5(1989); Hasegawa et al. J Carbohydrate Chem. 10:439-459(1991); Schwarzmann and Sandhoff, Meth. Enzymol. 138:319-341 (1987); Guadino and Paulson, J. Am. Chem. Soc. 116:1149-1150 (1994) (including supplemental material, which is also incorporated herein by reference). For example, the fatty acid moiety may be removed by base hydrolysis. Once the glycosylation reactions are completed, the same or a different fatty acid may be attached to the product of the glycosylation reactions.

Methods for coupling a fatty acid are generally known in the art and examples are discussed herein. The N-acyl group (NHR) of the compound of formula I, II, III, IV, or V may be derived from a wide variety of polyunsaturated fatty acids (or corresponding activated derivative, e.g., active ester, acid halide, etc.). Acylation may be carried out in the conventional way, for example, by reacting the starting products with an acylating agent, particularly with a reactive functional derivative of the acid, whose residue is to be introduced. Exemplary reactive functional derivatives of the acid include halides, anhydrides, and active esters. The acylation may be carried out in the presence of a base, (e.g., TEA, pyridine or collidine). Acylation is optionally carried out under anhydrous conditions, at room temperature or with heating. The Schotten-Baumann method may also be used to effect acylation under aqueous conditions in the presence of an inorganic base. In some cases it is also possible to use the esters of the acids as reactive functional derivatives. For acylation, it is possible to also use methods involving activated carboxy derivatives, such as are known in peptide chemistry, for example using mixed anhydrides or derivatives obtainable with carbodiimides or isoxazole salts.

Exemplary methods of acylation include: (1) reaction of the lysoglycosphingolipid derivative with the azide of the acid; (2) reaction of the lysoglycosphingolipid derivative with an acylimidazole of the acid obtainable from the acid with N, N′-carbonyldiimidazole; (3) reaction of the lysoglycosphingolipid derivative with a mixed anhydride of the acid and of trifluoro-acetic acid; (4) reaction of the lysoglycosphingolipid derivative with the chloride of the acid; (5) reaction of the lysoglycosphingolipid derivative with the acid in the presence of a carbodiimide (such as dicyclohexylcarbodiimide) and optionally of a substance such as 1-hydroxybenzotriazole; (6) reaction of the lysoglycosphingolipid derivative with the acid by heating; (7) reaction of the lysoglycosphingolipid derivative with a methyl ester of the acid at a high temperature; (8) reaction of the lysoglycosphingolipid derivative with a phenol ester of the acid, such as an ester with para-nitrophenol; and (9) reaction of the lysoglycosphingolipid derivative with an ester derived from the exchange between a salt of the acid and 1 methyl-2-chloropyridine iodide or similar products.

The invention also provides methods to prepare metal or organic base salts of the glycosphingolipid compounds for use according to the present invention having free carboxy functions, and these also form part of the invention. It is possible to prepare metal or organic base salts of other derivatives of the invention too, which have free acid functions, such as esters or peracylated amides with dibasic acids. Also forming part of the invention are acid addition salts of glycosphingolipid derivatives, which contain a basic function, such as a free amino function, for example, esters with aminoalcohols. Of the metal or organic base salts particular mention should be made of those which may be used in therapy, such as salts of alkali or alkaline earth metals, for example, salts of potassium, sodium, ammonium, calcium or magnesium, or of aluminum, and also organic base salts, for example of aliphatic or aromatic or heterocyclic primary, secondary or tertiary amines, such as methylamine, ethylamine, propylamine, piperidine, morpholine, ephedrine, furfurylamine, choline, ethylenediamine and aminoethanol. Of those acids which can give acid addition salts of the glycosphingolipid derivatives according to the invention special mention should be made of hydroacids such as hydrochloric acid, hydrobromic acid, phosphoric acid, sulfuric acid, lower aliphatic acids with a maximum of 7 carbon atoms, such as formic, acetic or propionic acids, succinic and maleic acids. Acids or bases, which are not therapeutically useful, such as picric acid, may be used for the purification of the glycosphingolipid derivatives of the invention and also form part of the invention.

Another suitable method of synthesizing a glycolipid of formula I, II, III, IV or V can use wild type or mutant endoglycoceramidase as taught in WO/2005/118798 which is incorporated herein by reference in its entirety with respect to the methods of synthesis and enzymes used therein. A donor substrate comprising a saccharide moiety and an acceptor substrate are contacted with a wild type or a mutant endoglycoceramidase having a modified nucleophilic carboxylate residue (i.e., Glu or Asp), wherein the nucleophilic Glu/Asp resides within a (Ile/Met/Leu/Phe/Val)-(Leu/Met/Ile/Val)-(Gly/Ser/Thr)-(Glu/Asp)-(Phe/Thr/Met/Leu)-(Gly/Leu/Phe) sequence of a corresponding wild-type endoglycoceramidase, under conditions wherein the endoglycoceramidase catalyzes the transfer of a saccharide moiety from a donor substrate to an acceptor substrate, thereby producing the glycolipid or aglycone.

Wild-type and mutant endoglycoceramidase polypeptides may be used to make glycolipid products in in vitro reactions mixes or by in vivo reactions, e.g., by fermentative growth of recombinant microorganisms that comprise nucleotides that encode endoglycoceramidase polypeptides.

Upon identifying a mutant endoglycoceramidase that is synthetically active, this enzyme may be used for production of a large variety of glycolipids of formula I, II, III, IV, or V, based on different combinations of heteroalkyl substrates. Many gangliosides of interest are described in Oettgen, H. F., ed., Gangliosides and Cancer, VCH, Germany, 1989, pp. 10-15, and references cited therein. Exemplified ganglioside end products include those listed in Table 2, below.

TABLE 2
Exemplified Ganglioside Formulas and Abbreviations

| Structure | Abbreviation |
|---|---|
| Neu5Ac3Gal4GlcCer | GM3 |
| GalNAc4(Neu5Ac3)Gal4GlcCer | GM2 |
| Gal3GalNAc4(Neu5Ac3)Gal4GlcCer | GM1a |
| Neu5Ac3Gal3GalNAc4(Neu5Ac3)Gal4GlcCer | GD1a |
| Neu5Ac3Gal3(Neu5Ac6)GalNAc4Gal4GlcCer | GD1α |
| Gal3GalNAc4(Neu5Ac8Neu5Ac3)Gal4GlcCer | GD1b |

Nomenclature of Glycolipids, IUPAC-IUB Joint Commission on Biochemical Nomenclature (Recommendations 1997); Pure Appl. Chem. (1997) 69:2475-2487; Eur. J. Biochem (1998) 257:293-298) (see, the worldwide web at chem.qmw.ac.uk/iupac/misc/glylp.html).

Further modifications may be made to the glycolipids synthesized using the endoglycoceramide synthase of the present invention. Exemplary methods of further elaborating glycolipids produced using the present invention are set forth in WO 03/017949; PCT/US02/24574; US2004063911 (although each is broadly directed to modification of peptides with glycosyl moieties, the methods disclosed therein are equally applicable to the glycolipids and method of producing them set forth herein). Moreover, the glycolipid compositions of the invention may be subjected to glycoconjugation as disclosed in WO03/031464 and its progeny (although each is broadly directed to modification of peptides with glycosyl moieties, the methods disclosed therein are equally applicable to the glycolipids and method of producing them set forth herein).

In yet another embodiment, the invention provides a pharmaceutical formulation comprising a glycolipid of Formula I, II, III, IV, or V or of appendices A through C in admixture with a pharmaceutically acceptable carrier. The pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions may be administered by a number of routes, for instance, the parenteral, subcutaneous, intravenous, intranasal, topical, oral or local routes of administration, such as by aerosol or transdermally, for prophylactic and/or therapeutic treatment. Commonly, the pharmaceutical compositions may be administered parenterally, e.g., intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

Parenteral vehicles include sodium chloride solution, Ringer's dextrose, dextrose and sodium chloride, lactated Ringer's, or fixed oils, intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like. The compositions may contain pharmaceutically acceptable auxiliary substances as required to approximate physiological conditions, such as pH adjusting and buffering agents, tonicity adjusting agents, wetting agents, detergents and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11. In an embodiment, the pH is from 5 to 9. In another embodiment, the pH is from 7 and 8.

The compositions containing the glycolipid compounds may be administered for prophylactic and/or therapeutic treatments. In therapeutic applications, compositions are administered to a subject already suffering from a disease, as described above, in an amount sufficient to cure or at least partially arrest the symptoms of the disease and its complications. Amounts effective for this use will depend, as discussed further below, on the particular compound, the severity of the disease and the weight and general state of the subject, but generally range from about 0.5 mg to about 2,000 mg of substrate per day for a 70 kg subject, with dosages of from about 5 mg to about 200 mg of the compounds per day being more commonly used.

In prophylactic applications, compositions containing the compound for use according to the invention are administered to a subject susceptible to or otherwise at risk of a particular disease. Such an amount is defined to be a “prophylactically effective dose.” In this use, the precise amounts again depend on the subject's state of health and weight, but generally range from about 0.5 mg to about 2,000 mg per 70 kilogram subject, more commonly from about 5 mg to about 200 mg per 70 kg of body weight.

Single or multiple administrations of the compositions may be carried out with dose levels and pattern being selected by the treating physician. In any event, the pharmaceutical formulations should provide a quantity of the substrates of this invention sufficient to effectively treat the subject.

Labeled substrates may be used to determine the locations at which the substrate becomes concentrated in the body due to interactions between the desired oligosaccharide determinant and the corresponding ligand. For this use, the compounds may be labeled with appropriate radioisotopes, for example, ¹²⁵I, ¹⁴C, or tritium, or with other labels known to those of skill in the art.

The dosage ranges for the administration of the compounds for use according to the invention are those large enough to produce the desired effect in which the symptoms of the deficiency state show some degree of suppression. Generally, the dosage will vary with the age, condition, sex and extent of the disease in the subject and may be determined by one of skill in the art. The dosage may be adjusted by the individual physician in the event of any counter indications.

Additional pharmaceutical methods may be employed to control the duration of action. Controlled release preparations may be achieved by the use of polymers to conjugate, complex or adsorb the glycosphingolipid. The controlled delivery may be exercised by selecting appropriate macromolecules (for example, polyesters, polyamino carboxymethylcellulose, and protamine sulfate) and the concentration of macromolecules as well as the methods of incorporation in order to control release. Another possible method to control the duration of action by controlled release preparations is to incorporate the glycosphingolipid into particles of a polymeric material such as polyesters, polyamino acids, hydrogels, poly (lactic acid) or ethylene vinylacetate copolymers. In one embodiment, the compositions provide a controlled release of an oral administered composition in the lower GI tract or intestines.

In order to protect the compounds for use according to the invention from binding with plasma proteins, the compounds may be entrapped in microcapsules prepared, for example, by coacervation techniques or by interfacial polymerization, for example, hydroxymethylcellulose or gelatin-microcapsules and poly (methyl methacrylate) microcapsules, respectively, or in colloidal drug delivery systems, for example, liposomes, albumin microspheres, micro emulsions, nanoparticles, and nanocapsules or in macro emulsions. Such teachings are disclosed in Remington's Pharmaceutical Sciences (16th Ed., A. Oslo, ed., Mack, Easton, Pa., 1980).

In an exemplary embodiment, the compounds for use according to the invention are formulated as components or contents of a liposome, used as a targeted delivery system. When phospholipids are gently dispersed in aqueous media, they swell, hydrate, and spontaneously form multilamellar concentric bilayer vesicles with layers of aqueous media separating the lipid bilayer. Such systems are usually referred to as multilamellar liposomes or multilamellar vesicles (MLVs) and have diameters ranging from about 100 nm to about 4 microns. When MLVs are sonicated, small unilamellar vesicles (SUVS) with diameters in the range of from about 20 to about 50 nm are formed, which contain an aqueous solution in the core of the SUV.

Examples of lipids useful in liposome production include phosphatidyl compounds, such as phosphatidylglycerol, phosphatidylcholine, phosphatidylserine, and phosphatidylethanolamine. Particularly useful are diacylphosphatidylglycerols, where the lipid moiety contains from 14-18 carbon atoms, particularly from 16-18 carbon atoms, and are saturated. Illustrative phospholipids include egg phosphatidylcholine, dipalmitoylphosphatidylcholine, and distearoylphosphatidylcholine.

In preparing liposomes containing the compounds for use according to the invention, such variables as the efficiency of compound encapsulation, lability of the compound, homogeneity and size of the resulting population of liposomes, compound-to-lipid ratio, permeability instability of the preparation, and pharmaceutical acceptability of the formulation should be considered. Szoka, et al, Annual Review of Biophysics and Bioengineering, 9:467 (1980); Deamer, et al., in LIPOSOMES, Marcel Dekker, New York, 1983, 27; Hope, et al., Chem. Phys. Lipids, 40:89 (1986)).

A compound for use according to the invention, alone or as part of a pharmaceutical composition, may be sterilized prior to administration. Sterility may be readily accomplished by filtration through sterile membranes such as 0.2 micron membranes, or by other conventional methods. A compound of the invention, alone or as part of a pharmaceutical composition, typically may be stored in lyophilized form or as an aqueous solution. pH may be a factor for certain modes of administration. In such instances, the pH typically will range between about 2 to 10, for example, between about 5 to 8, and in a particular embodiment, 6.5 to 7.5. In an embodiment, the pH is physiological pH.

Subjects (i.e., mammals, including humans) in need of treatment may be administered a therapeutically effective amount or dosage, i.e., an amount or dosage of a compound of the invention, alone or as part of pharmaceutical composition, that will provide efficacy in treating the ganglioside deficiency state, disorder or condition to be treated. As would be recognized by those of skill in the art, a “therapeutically effective amount” will vary with the mode of administration and subject thus may be determined on a case by case basis. Factors to be considered include, but are not limited to, the subject (e.g. mammal) being treated, its sex, weight, diet, concurrent medication, overall clinical condition, the particular compounds employed, and the specific use for which these compounds are employed. Therapeutically effective amounts or dosages may be determined by either in vitro or in vivo methods. In general, a “therapeutically effective amount” or “therapeutically effective dose” of a compound or composition is an amount or dose that will result in the prophylaxis, treatment, amelioration, reduction or cure of one or more of nervous system impairments associated with the deficiency state, disorder or condition.

Modes of administration include those known in the art including, but not limited to, oral, injection, intravenous (bolus and/or infusion), topical, subcutaneous, intracerebroventricular, intrathecal, intramuscular, colonic, rectal, nasal and intraperitoneal administration. The administration may further be local or systemic. In an embodiment, compounds for use according to the invention, alone or as part of a pharmaceutical composition are taken orally. In one embodiment, the compositions are formulated for release in the intestine upon oral administration.

For injection by hypodermic needle, it may be assumed the dosage is delivered into the body's fluids. For other routes of administration, the absorption efficiency may be individually determined for each compound of the invention by methods well known in pharmacology. Accordingly, as would be understood by one of skill in the art, it may be necessary for the therapist to titer the dosage and modify the route of administration as required to obtain the optimal therapeutic effect. The determination of effective dosage levels, that is, the dosage levels necessary to achieve the desired result, will be within the ambit of one skilled in the art. Typically, a compound of the invention is administered at lower dosage levels, with dosage levels being increased until the desired effect is achieved.

The pharmaceutical compositions and formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In a specific example, treatment comprises administration of a therapeutically effective amount of a pharmaceutical composition comprising GM1 to the central nervous system (CNS) of a subject.

In a specific example, treatment provides GM1 to the tissues of the CNS by administration directly into the cerebrospinal fluid (CSF). Means of delivery to the CSF and brain include, but are not limited to intrathecal (IT), intracerebroventricular (ICV), and intraparenchymal administration. IT or ICV administration may be achieved through the use of surgically implanted pumps that infuse the therapeutic agent into the cerebrospinal fluid.

Intraparenchymal delivery may be achieved by the surgical placement of a catheter into the brain. As used herein, “delivery to the CSF” and “administration to the CSF” encompass the IT infusion or ICV infusion of GM1 through the use of an infusion pump. In some embodiments, IT infusion is a suitable means for delivery to the CSF. In other embodiments, GM1 is continuously infused into the CSF for the entire course of treatment; such administration is referred to as “continuous infusion” or, in the case of IT infusion, “continuous IT infusion.” Also contemplated is continuous intraparenchymal infusion using a pump.

The solutions and compositions used in the present invention may be delivered to the brain by any known means. In one embodiment, the composition may be delivered by way of a catheter or other delivery device having one end implanted in a tissue, e.g., the brain by, for example, intracranial infusion. In some aspects, intracranial infusion is intrastriatal infusion, intraputamenal infusion, intracaudate infusion, intraventricular infusion, intraparenchymal infusion and intracortical infusion. In another aspect, a solution or composition is delivered to the CNS by intrathecal infusion.

An embodiment of the invention provides a system for determining and delivering to the brain of a mammal a solution or composition. The solution or composition may comprise one or more of an iRNA agent, a tracer, a therapeutic molecule or an imaging agent.

Systems useful for delivering solutions or compositions to the brain are described in US Published Application No. 2005/0048641 and US Published Application No. 2012/0116306. The system comprises a therapy delivery device composed of a pump coupled to a reservoir for housing a solution or composition. The system further comprises a catheter. The catheter comprises a proximal end coupled to the pump and a delivery region adapted for delivering the composition or solution to a delivery location within the mammal. The catheter can have one or more delivery regions along the length of the catheter and that a delivery region may or may not be at the distal end of the catheter. The therapy delivery device may be implantable or may be an external device. The therapy delivery device can have a port into which a hypodermic needle may be inserted to inject a quantity of solution or composition into reservoir. The device can further comprise a catheter port, to which the proximal end of catheter may be coupled. The catheter port in this embodiment is operably coupled to pump. A connector may be used to couple the catheter to the catheter port of the device. Device can take the form of any pump system, including but not limited to, a drug reservoir and/or a drug pump of any kind, for example an osmotic pump, an infusion pump, an electromechanical pump, an electroosmotic pump, an effervescent pump, a hydraulic pump, a piezoelectric pump, an elastomeric pump, a vapor pressure pump, or an electrolytic pump. One example of a suitable pump is the device shown in U.S. Pat. No. 4,692,147 (Duggan), assigned to Medtronic, Inc., Minneapolis, Minn., an embodiment of which is commercially available as the Synchromed® infusion pump manufactured by Medtronic, Inc., the patent is incorporated herein by reference. The device may also take the form of Medtronic's Synchromed® II infusion pump.

The therapy delivery device, such as Medtronic's SYNCHROMED or SYNCHROMED II pump systems, may be operated to discharge a predetermined dosage of the pumped fluid to a delivery location of a mammal. The therapy delivery device can contain a microprocessor or similar device that may be programmed to control the amount and/or rate of delivery of the composition or solution or programmed to change the concentration of a tracer, therapeutic composition or agent in the solution being delivered to the mammal. The programming may be accomplished with an external programmer/control unit (not shown) via telemetry. A controlled amount of a solution or composition comprising a therapeutic agent may be delivered over a specified time period. With the use of a therapy delivery device, different dosage regimens may be programmed for a particular mammal. A programmed therapeutic device allows for starting conservatively with lower doses and adjusting to a more aggressive dosing scheme, if warranted, based on safety and efficacy factors. It may be desirable to reduce, rather than eliminate, the temporal or spatial expression of a targeted gene in which case a therapy delivery device can allow for appropriate dose titration and distribution. Delivery device can also allow for delivery of therapeutic agent to be stopped temporarily and resumed when desired. For example, delivery of agent may be stopped to perform diagnostic tests, intervene with a different therapy, or for safety reasons.

The device may be implanted below the skin of a patient. In an embodiment, the device is implanted in a location where the implantation interferes as little as practicable with activity of the mammal. The device may be implanted subcutaneously in any medically acceptable area of the human body such as in a subcutaneous pocket located in the chest below the clavicle, in an abdominal subcutaneous pocket, in the patient's cranium, and the like.

In one embodiment, therapy delivery device and delivery system may take the form of a device and system described in U.S. Pat. No. 6,042,579, entitled “Techniques For Treating Neurodegenerative Disorders By Infusion Of Nerve Growth Factors Into The Brain”, which patent is incorporated herein by reference in its entirety.

The delivery system may include a sensor. Sensor may detect an event associated with an effect of compounds of formula (I) to (V), the disease to be treated, the distribution of a therapeutic agent in the brain or other change in a delivery parameter. The sensor may relay information regarding the detected event, in the form of a sensor signal, to processor of device. The sensor may be operably coupled to processor in any manner. For example, the sensor may be connected to processor via a direct electrical connection, such as through a wire or cable. Sensed information, whether processed or not, may be recorded by the device and stored in memory. The stored sensed memory may be relayed to an external programmer, where a physician may modify one or more parameter associated with the therapy based on the relayed information. Alternatively, based on the sensed information, the microprocessor may adjust one or more parameters associated with delivery of a solution or composition. For example, the microprocessor may adjust the amount and timing of the infusion of such solution or composition. Any sensor capable of detecting an event associated with an effect of the RNA inhibitory agent, the disease to be treated, the distribution of a therapeutic agent in the brain or other change in a delivery parameter may be used. In an embodiment, the sensor is implantable. It will be understood that two or more sensors may be employed.

In an embodiment, the sensor may be a sensor as described in, e.g., U.S. Pat. No. 5,978,702, entitled “Techniques Of Treating Epilepsy By Brain Stimulation And Drug Infusion,” which patent is hereby incorporated herein by reference in its entirety, or U.S. patent application Ser. No. 10/826,925, entitled “Collecting Sleep Quality Information Via A Medical Device,” filed Apr. 15, 2004, which patent application is hereby incorporated herein by reference in its entirety.

Methods that use a catheter to deliver a therapeutic agent to the brain generally involve inserting the catheter into the brain and delivering the agent, solution or composition to the desired location. To accurately place the catheter and avoid unintended injury to the brain, surgeons typically use stereotactic apparatus/procedures. (U.S. Pat. No. 4,350,159) During a typical implantation procedure, an incision may be made in the scalp to expose the patient's skull. After forming a burr hole through the skull, the catheter may be inserted into the brain.

Other delivery devices useful with methods of this invention includes a device providing an access port, which may be implanted subcutaneously on the cranium through which therapeutic agents may be delivered to the brain, such as the model 8506 ICV Access Port and the 8507 Intraspinal Port, developed by Medtronic, Inc. of Minneapolis, Minn. Two models of catheters that can function with the model 8506 access port include the model 8770 ventricular catheter (Medtronic, Inc.), for delivery to the intracerebral ventricles, which is disclosed in U.S. Pat. No. 6,093,180, and the infusion catheter developed by Medtronic, Inc., for delivery to the brain tissue itself (i.e., intraparenchymal delivery), described in U.S. patent publications 2009/540,444 and 2009/625,751, the teachings of which are herein incorporated by reference. The latter catheter has multiple outlets on its distal end to deliver the therapeutic agent to multiple sites along the catheter path. Other systems provide catheter assemblies, systems, and methods operable to introduce an agent into the body while reducing the occurrence of backflow of the agent along the catheter assembly track. (U.S. patent publication 2009/143,764) Such catheter assemblies may be used in various applications including the treatment of acute and chronic medical conditions.

The striatum is a suitable area of the brain for delivery of compounds of formula (I) to (V). Stereotactic maps and positioning devices are available and positioning may be effected by the use of anatomical maps obtained by CT and/or MRI imaging of the subject's brain to help guide the delivery device to the chosen target.

A therapeutic or prophylactic amount effective to treat a neurological disorder by the methods disclosed herein comprises a sufficient amount of compound formula (I) to (V) delivered during the entire course of treatment to ameliorate or reduce the symptoms of the neurological disorder being targeted for treatment. These compound formula (I) to (V) can also contain a pharmaceutically acceptable carrier or excipient. Such carriers or excipients include any pharmaceutical agent that does not itself induce the production of antibodies harmful to the individual receiving the composition, and which may be administered without undue toxicity. Additionally, compositions for intracranial administration can include sterile aqueous solutions which can also contain buffers, diluents and other suitable additives.

The therapeutic compositions of the invention are administered in dosages sufficient to treat Parkinson's disease. The methods of the invention contemplate that the concentration of the labeled therapeutic agent is in the range of 0.1 mg/mL to 200 mg/mL. In one embodiment, the concentration of therapeutic agent is 0.5 mg/mL to 50 mg/mL. In another embodiment, the concentration of therapeutic agent in the tracing composition is 2 mg/mL to 25 mg/mL.

The therapeutic agent and compositions may be administered by continuous delivery, intermittent delivery, or through a combination of continuous and intermittent delivery. In one embodiment, the composition is continuously administered from 1 hour to 30 years. In other embodiments, the composition is continuously administered from 6 hours to 20 years, or from 1 day to 35 days. In another embodiment, intermittent administration of the composition comprises: two or more cycles of administration, wherein one cycle is 1 hour to 30 days of continuous administration followed by 1 day to 60 days with no administration; two or more cycles of administration, wherein one cycle is 6 hours to 10 days of continuous administration followed by 1 day to 60 days with no administration; two or more cycles of administration, wherein one cycle is 6 hours to 3 days of continuous administration followed by 3 days to 30 days with no administration; or two or more cycles of administration, wherein one cycle is 1 day to 3 days of continuous administration followed by 4 days to 21 days with no administration. The time for the cycle of administration after the no administration term may be the same as, shorter than, or longer than the time of the administration cycle before the no administration term.

The composition may be administered continuously at a rate of 0.03 μL/min to 10 μL/min. In other embodiments, the composition is administered continuously at a rate of 0.05 μL/min to 1.0 μL/min. In another embodiment, the concentration of the therapeutic agent in the composition is 4 mg/mL to 200 mg/mL and is administered continuously at a rate of 0.03 μL/min to 2.0 μL/min for 2 or more days.

In one embodiment, the therapeutic agent inhibits α-synuclein protein by at least 20% within 4 mm from the site of administration. In another embodiment, the therapeutic agent inhibits α-synuclein protein by at least 40% within 4 mm from the site of administration.

In one embodiment, the therapeutic agent increases dopamine by at least 20% within 4 mm from the site of administration. In another embodiment, the therapeutic agent increases dopamine by at least 40% within 4 mm from the site of administration.

In another embodiment, the therapeutic agent increases dopamine transporters (e.g. DAT and/or VMAT) by at least 20% within 4 mm from the site of administration. In another embodiment, the therapeutic agent increases dopamine transporters by at least 40% within 4 mm from the site of administration.

Many factors can influence the dosage and timing required to effectively treat a subject, including, but not limited to, the severity of the disease or disorder, previous treatments, the general health and/or age of the subject, and other diseases present. Moreover, treatment of a subject with compounds of formula (I) to (V) or a therapeutically effective amount of a composition can include a single treatment or a series of treatments. Estimates of effective dosages and in vivo half-lives for the individual therapeutic agents encompassed by the invention may be made using conventional methodologies or on the basis of in vivo testing using an appropriate animal model. In addition, the label can dissociate from the therapeutic agent at a measured rate. If that occurs, the calculation of the effective dosage may be corrected such that actual tissue concentration is calculated.

In addition to their administration individually or as a plurality, therapeutic agents of the invention may be administered in combination with other known agents effective in treatment of diseases. In any event, the administering physician can adjust the amount and timing of the compounds of the invention by administrating on the basis of results observed using standard measures of efficacy known in the art or described herein.

The present invention provides treatment compositions and systems and methods for determining and delivering an effective amount of treatment composition for treating a central nervous system disorder in a mammal. Determining the effective amount of a treatment composition to be delivered to the CNS is desirable because if too little therapeutic agent is administered, the disease is not treated. If too much therapeutic agent is administered, the therapeutic is wasted, a consequence which is particularly undesirable when the therapeutic agent is expensive or difficult to obtain.

In the present methods, a tracing composition is administered, via a catheter, to the brain of a mammal. The tracing composition contains a detectably labeled therapeutic agent. The detectable label does not adversely affect the effectiveness of the labeled therapeutic agent as compared to an therapeutic agent without the label. Additionally, the detectable label that does not adversely affect the distribution of the labeled therapeutic agent as compared to the labeled therapeutic agent without the label. The labeled therapeutic agent used in tracing composition may be any of the compositions above.

When observing distribution of an infused drug to the brain, typically the drug distribution is referred to as the “Volume of Distribution” or V_{d}. The V_{d} is a measure of the distribution of the label, and the sensitivity of the instrument to detect the label, rather than a measurement of where the drug is having an effect. Also of interest to researchers and clinicians is where the drug is having a meaningful physiological effect. This is the Volume of Efficacy, Vₑ, which is defined as the volume of the brain where the drug concentration is higher than the Effective Concentration (cₑ). The cₑ can also be determined from any drug distribution imaging modality and its corresponding efficacy modality. For example, the cₑ may be determined using quantitative autoradiography (qARL) and brain imaging (e.g. PET, SPECT an MRI) to image the distribution of the drug after an infusion.

Accordingly, a solution comprising a tracer is introduced via a catheter to the brain of a mammal, and the distribution of the solution during delivery is monitored by imaging the tracer in the solution to determine whether a target volume of distribution at steady state is substantially achieved.

If a target volume of distribution at steady state is not substantially achieved, the rate of delivery of the solution and/or the concentration of the tracer in the solution is modified until target volume of distribution at steady state is substantially achieved. The rate of delivery of the therapeutic composition and concentration of the therapeutic agent in the therapeutic concentration to substantially achieve the target volume of distribution at steady state can then be determined based on the rate of delivery and concentration of the tracer solution at which the target volume of distribution at steady state was substantially achieved. The therapeutic composition can then be delivered via a catheter to the brain of the mammal using the rate and concentration determined from the delivery of the solution containing the tracer.

The solution may be delivered by convection enhanced delivery (CED). CED of drugs directly into the parenchyma of the brain uses a continuous pressure gradient for distribution of drug into the interstitial space. The primary advantages of CED are circumvention of the blood-brain barrier by delivering drug directly to the extracellular space of the cells of interest, and the possibility of achieving broad distribution of drug with CED flow rates. To date, CED has been used clinically for administering drugs directly to the CNS to treat glioblastoma and Gaucher disease; however, there is potential for much broader applicability to treat neurological disorders, including neurodegenerative diseases such as Parkinson's disease.

Monitoring the distribution of the solution and imaging the tracer through the brain tissue may be done by any imaging technique such as, for example, magnetic resonance imaging (MRI) or X-ray, e.g. positron emission topography (PET), and computed tomography (CT). If the tracer may be assumed to, or is known to have mobility in the brain tissue that is substantially similar to the therapeutic agent, delivery may be ceased when the tracer is observed to reach a target volume of distribution at steady state. Delivery may also be ceased before target volume of distribution at steady state is achieved by the tracer if it is expected or known that that the therapeutic agent has a greater mobility in the tissue than the tracer. For example, where a correlation has been established between the mobilities of the tracer and the therapeutic agent, delivery may be ceased when the observed distribution of the tracer corresponds to a desired distribution of the therapeutic agent.

If the tracer does not have a mobility that is substantially similar to the therapeutic agent, or cannot be assumed to have a substantially similar mobility as the therapeutic agent (for example, because the agent is highly toxic and delivery of the agent will damage sensitive issues such as brain tissue outside of the target tissue) the volume of steady state distribution of the tracer that is observed may be converted to a volume of distribution of the therapeutic agent using a previously established correlation between the two. Thus, monitoring the volume of distribution for the tracer may be used to determine if the therapeutic agent has reached the target volume of distribution at steady state in the target tissue.

A tracer may comprise a metal chelate. A metal chelate is a complex of a metal ion and a metal chelating group (a group of atoms that serves to bind the metal ion). Examples of metal chelating groups include natural and synthetic amines, porphyrins, aminocarboxylic acids, iminocarboxylic acids, ethers, thiols, phenols, glycols and alcohols, polyamines, polyaminocarboxylic acids, polyiminocarboxylic acids, aminopolycarboxylic acids, iminopolycarboxylic acids, nitrilocarboxylic acids, dinitrilopolycarboxlic acids, polynitrilopolycarboxylic acids, ethylenediaminetetracetates, diethylenetriaminepenta or tetraacetates, polyethers, polythiols, cryptands, polyetherphenolates, polyetherthiols, ethers of thioglycols or alcohols, polyaminephenols, all either acyclic, macrocyclic, cyclic, macrobicyclic or polycyclic, or other similar ligands which produce stable metal chelates or cryprates (including sepulchrates, sacrophagines, and crown ethers). Specific examples of metal chelating groups include 1,4,7,10-tetraazacyclododecanetetraacetic acid (DOTA), 1,4,7,10-tetraazacyclododecane-1,4,7-triacetic acid (DO3A), 1-oxa-4,7,10-triazacyclododecane-triacetic acid (DOXA), 1,4,7-triazacyclononanetriacetic acid (NOTA), 1,4,8,11-tetraazacyclotetradecanetetraacetic acid (TETA), DOTA-N(2-aminoethyl)amide and DOTA-N-(2-aminophenethyl)amide, BOPTA, HP-DO3A, DO3MA, DTPA, and various derivatives thereof. Additional examples are provided in Caravan et al., 1999, Chem. Rev., 99:2293-2352 and in U.S. Pat. Nos. 5,246,692, 5,292,868 and 5,434,287. In one embodiment, the metal chelate is 2-(p-isothiocyanatobenzyl)-6-methyl diethylenetriamine pentaacetic acid (1B4M) chelates of gadolinium (III) ion.

Metals ions of the metal chelates may be paramagnetic ions if the imaging agent is to be used as a MRI contrast agent. Suitable metal ions include those having atomic numbers of 22-29 (inclusive), 42, 44 and 58-70 (inclusive) and have oxidation states of +2 or +3. Examples of such metal ions are chromium (III), manganese (II), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III).

If the macromolecular imaging agent is to be used as an X-ray contrast agent, the metal ion may be selected from the ions of W, Bi, Hg, Os, Pb, Zr, lanthanides, and combinations thereof. If a combined MRI/X-ray contrast agent is desired, the metal ion may be selected from the paramagnetic lanthanide ions. If a radiographic imaging agent is desired, the metal may be radioactive, such as the radioactive isotopes of In, Tc, Y, Re, Pb, Cu, Ga, Sm, Fe, or Co.

In other embodiments, the tracer comprises an iodinated CT contrast agent, such as iopanoic acid or iopamidol.

The tracer may be conjugated to a therapeutic agent. Although conjugation typically refers to formation of a covalent bond between the metal chelate and the therapeutic agent, other types of bonds (for example, ionic, dipole-dipole, or van der Waals) may suffice in some embodiments. The therapeutic agent of the present invention can include, but are not limited to, antisense oligonucleotides, ribozymes, iRNA, proteins, drugs, antibodies, antibody fragments, immunotoxins, chemical compounds, protein fragments and toxins.

Regardless of whether or not the tracer contains a therapeutic agent, the tracer is delivered in an amount sufficient to produce a target volume of distribution at steady state of the tracer solution determined by the image intensity of the tracer in the brain tissue. For example, if MRI is used to image the solution, the distribution of the tracer solution may be detected at some time after the imaging agent is administered. Monitoring the distribution of the solution can occur over a plurality of time intervals.

The present invention also contemplates methods of monitoring the effectiveness of a therapeutic composition containing a therapeutic agent delivered via a catheter to the brain of a mammal. This monitoring can occur after treatment with a therapeutic composition has started. The method can include the steps of: (a) after administering the therapeutic composition at a rate and concentration of the therapeutic agent in the composition to substantially achieve a target volume of distribution at steady state, (b) delivering a solution comprising a tracer via a catheter to the brain of a mammal to mimic the conditions at which the therapeutic composition was administered; and (c) monitoring distribution of the solution during delivery by imaging the tracer in the solution to determine whether a target volume of distribution at steady state is substantially achieved. Additional steps in the method can include: (d) if the target volume of distribution at steady state is not substantially achieved in step (c), modifying the rate of delivery of the solution or the concentration of the tracer in the solution or both, until the target volume of distribution at steady state is substantially achieved; (e) determining a rate of delivery of the therapeutic composition and a concentration of the therapeutic agent in the therapeutic concentration to substantially achieve the target volume of distribution at steady state based on the rate of delivery and concentration at which the tracer solution substantially achieved the target volume of distribution at steady state in step (c) or (d); and delivering the therapeutic composition at the rate determined in step (e) with the concentration of therapeutic agent in the therapeutic composition determined in step (e) via a catheter to the brain of the mammal. It is also contemplated that if the target volume of distribution at steady state is not achieved in step (c) or in step (d), then step (e) is performed at a predetermined rate and concentration of therapeutic agent.

In some embodiments, an infusion pump is employed to deliver GM1 to the CNS. Such infusion pumps and their method of implantation and use are known to the skilled worker. In a specific example, the Medtronic SyncroMed® II pump, is employed to deliver GM1 to the CNS. The SyncroMed® II pump is surgically implanted according the procedures set forth by the manufacturer. The pump contains a reservoir for retaining a drug solution, which is pumped at a programmed dose into a catheter that is surgically implanted.

A typical dosage might range from about 0.01 mg/kg to about 1000 mg/kg, for example, from 0.1 mg/kg to about 100 mg/kg, from about 0.1 mg/kg to about 30 mg/kg. In an embodiment, the dosage range is from about 0.1 mg/kg to about 10 mg/kg. In an embodiment, the dosage range is from 0.1 mg/kg to about 3 mg/kg. Advantageously, the compounds for use according to the invention, alone or as part of a pharmaceutical composition, maybe administered several times daily, and other dosage regimens may also be useful. A compound of the invention may be administered on a regimen in a single or multidose (e.g. 2 to 4 divided daily doses) and/or continuous infusion. The regimen may be tailored over time according to the response of the subject. The dosage regimen may be daily, weekly, monthly, acute, subacute, subchronic or chronic. Up to 30 g (e.g., 10 to 100 mg, 1 to 2, 2 to 5, 5 to 10, 10 to 20, or 20 to 30 g), for instance, may be administered in one dose.

The effectiveness of the compounds for use according to the invention may be determined using screening protocols known in the art. The biological properties of the compounds for use according to the invention may be readily characterized by methods that are well known in the art including, for example, in vitro screening protocols (e.g. cell culture (MPTP (rat ventral mesophenthalic cells), NMDA (mouse primary cortical neurons), ceramide (neuroblastoma-human)), CACO-2 (oral absorption), RBC lysis) and in vivo studies (e.g. mouse and primate MPTP toxicity studies (IP, IV, and/or oral) for effectiveness. Exemplary methods for identifying or screening suitable compounds for use according to the invention are set forth below.

In some embodiments, the subject to be treated with a compound according to the invention is diagnosed as having a ganglioside deficiency by having a sample from the subject analyzed for its ganglioside content and then a compound of the invention having a saccharide moiety of the deficient ganglioside or a ganglioside downstream thereof is administered to the subject to treat the subject. In some further embodiments, the deficient ganglioside is GM1 and/or a ganglioside downstream of GM1.

### Examples

### Example 1

### Enzymatic Synthesis of Lyso-GM1 by Mutant EGC Enzymes

Reactions were performed in 25 mM NaOAc (pH 5.0) containing 0.1-0.2% Triton X-100. A typical reaction mixture contained approximately 50 mg/mL of a fluorinated GM1 sugar donor (GM1-F), 15 mg/ml of an acceptor sphingosine, and 2.0 mg/ml of the appropriate EGC mutant in a total reaction volume of 50 μL. Under these conditions, the reaction proceeds to >90% completion within 12 hours at 37° C. based on TLC analysis.

Transfer of the fluorinated GM1 sugar donor was monitored using an HPLC reverse phase method on a Chromolith RP-8e column with eluants of 0.1% trifluoroacetic acid (TFA) in acetonitrile (ACN) to 0.1% TFA in H₂O. The lyso-GM1 was purified using silica gel chromatography.

### Example 2

### General Procedure for Acylating Lyso-GM1

Lyso-GM1 was dissolved in methanol-DMF and triethyl amine (5 mol eq) and fatty acid anhydride (3 mol eq) were added. After stirring overnight, the solution was concentrated to dryness and the acylated GM1 purified by silica gel chromatography.

### Example 3

### General Procedure for Preparing the GM1 Aldehyde

GM1 (2.5 g, 1.62 mmol) was dissolved in 2500 mL of methanol. This solution was cooled to −70° C. and ozone bubbled through the solution until the light blue color did not disappear (about 30 mins). The ozone was removed by bubbling nitrogen through the reaction mixture until the solution became colorless. Then, 80 mL of dimethylsulfide was added and the resulting mixture was stirred at room temperature for 2 h. The solvent was evaporated with nitrogen to dryness. The residue was co-evaporated with toluene (50 mL) and the residue dried on a high vacuum pump for 1 hr to yield a white solid containing the aldehyde.

### Example 4

### Preparation of Compounds (XXVII) and (XXVIII); General Reaction Conditions for Preparing Compounds of Formula (I), (III), and (IV)

A suspension containing the Wittig salt such as 3-chloro-2-fluoro-5-(trifluoromethyl)benzyl-triphenylphosphonium bromide (2.58 g, 4.66 mmol), dimethylformamide (DMF) (50 mL) was cooled to −40° C. and 1M potassium tert-butyloxide in tert-butylalcohol solution (4.49 mL) was then added. After 10 minutes, this reaction mixture was added slowly to a solution of aldehyde dissolved in DMF (200 mL) and cooled to −40° C. After addition was complete, the reaction mixture was stirred at room temperature for 1 h. The reaction mixture was then concentrated on a rotovap and the residue chromatographed (silica, CHCl₃/MeOH 3:1 then, MeOH/H₂O/NH₄OH 60:40:7:1) to afford 1.5 g (60% yield) of the compounds (XXVII) and XXVIII) as a 70/30 cis/trans mixture. ESI-MS; calcd for C67H106ClF4N3O31, 1559. found 1558 [M-1]; 1H-NMR (500 MHz, 95% DMSO-d6+5% D2O) ∂ 7.98 (d, J 6.0 Hz, 2H), 7.84 (d, J 6.0 Hz, 1H), 7.82 (d, J 5.5 Hz, 2H), 7.60 (d, J 5.5 Hz, 1H), 7.34 (d, J 9.5 Hz, 2H), 6.64 (d, J 16 Hz, 1H), 6.48 (d, J 11.5 Hz, 2H), 5.93 (dd, J 11.5/11.5 Hz, 2H), 4.79 (d, J 8.5 Hz, 2H), 4.27 (d, J 8.0 Hz, 2H), 4.21 (d, J 8.5 Hz, 2H), 3.00-4.00 (m), 1.98 (m, 2H), 1.86 (s, 3H, COCH3), 1.78 (s, 3H, COCH3), 1.25 (m), 0.83 (t, 3H, CH3).

### Example 5

### Preparation of Compounds (XXXVII) and XXXVIII). General Reaction Conditions for Preparing Compounds of Formula (II)

The GM1 aldehyde (20 mg, 0.013 mmol) and primary or secondary amine (e.g. dioctylamine; 6 mg, 0.024 mmol), were added with stirring to 2.5 mL of dimethylformamide (DMF) at room temperature. Then an organoboronate or boronoic acid (e.g. trans-2-phenylvinylboronic acid; 9 mg, 0.045 mmol) in methanol (5 mL) was added. The resulting solution was stirred at room temperature for three days. The reaction mixture was then concentrated to dryness on a rotovap and the residue purified by solid phase extraction using a 1 g HAX cartridge. The eluant was then purified using HPLC to afford 9.5 mg (43% yield) of white solid, compound of formula (XXVII) and (XXXVIII). ESI-MS; calcd for C83H144N4O31, 1693. found 1692 [M-1]; 1H-NMR (500 MHz, 95% DMSO-d6+5% D20) ∂ 8.05 (d, J 3.0 Hz, 1H), 7.70 (m, 5H), 6.40 (m, 1H), 6.25 (dd, J 9.0 and 16 Hz, 1H), 4.80 (d, J 8.5 Hz, 1H), 4.28 (d, J 8.0 Hz, 1H), 4.22 (d, J 8.0 Hz, 1H), 4.16 (d, 4.2 Hz, 1H), 3.00-4.00 (m), 2.10 (m, 2H), 1.86 (s, 3H, COCH3), 1.60 (s, 3H, COCH3), 1.19 (s), 0.83 (t, 3H, CH3).

### Example 6

### Protection of Cortical Cells from Apoptosis

To induce apoptosis, rat cortical cells were cultured and treated with 50 μM hydrogen peroxide for three hours prior to being treated with the ganglioside analogue. The cells were also treated with the hydrogen peroxide during treatment with the ganglioside analogue and post-treatment for 48 h. Cell death was assayed using the MTT assay.

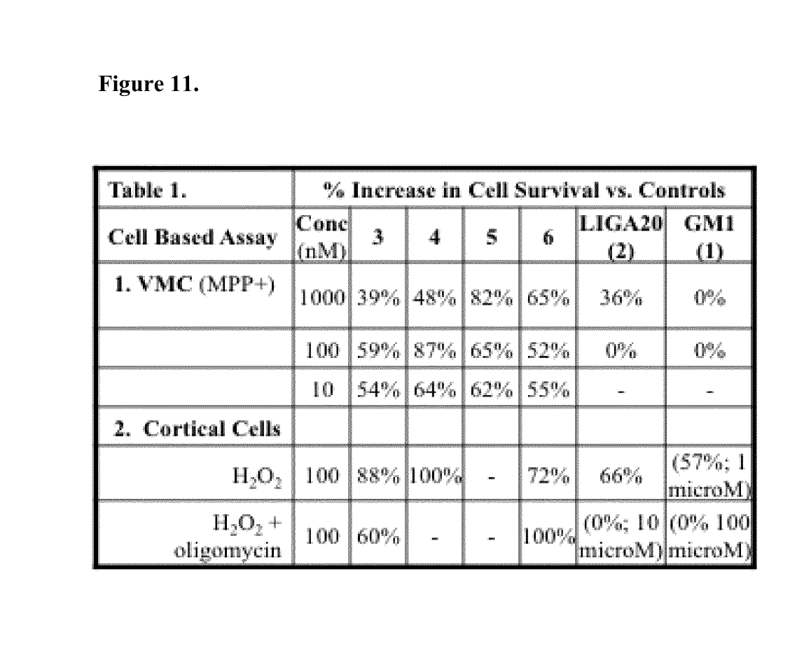

Approximately 30% of the cells treated with hydrogen peroxide died as a result of the treatment. Treatment with GM1 or compounds of the invention provided protection of the cells from apoptosis (FIG. 11).

### Example 7

### Protection of Cortical Cells from Cell Death

To induce non-apoptotic cell death, rat cortical cells were cultured and treated with 50 μM hydrogen peroxide and oligomycin (0.01 μM) for three hours prior to being treated with the compounds of the invention. The cells were treated with the hydrogen peroxide and oligomycin during treatment with the ganglioside analogue and post-treatment for 48 h. Cell death was assayed using the MTT assay.

Approximately 30% of the cells treated with hydrogen peroxide die as a result of the treatment. Treatment of the cells with compounds for use according to the invention protected cells from death (FIG. 11).

### Example 8

### Protection of Dopaminergic Cells from Cell Death

Rat striatal dopaminergic cells (VNC) were cultured and treated with MPP for 24 hrs. The cells were then treated with compounds of the invention at varied dosing, incubated for 24 hrs and cell the number of tyrosine hydroxylase (TH+) immunostained cells determined. Treatment of the cells with compounds for use according to the invention protected cells from death (FIG. 11).

### Example 9

### Rescue of Striatal Dopamine Levels and Dopaminergic Cells in MPTP-Treated Mice

C57B1/6 mice 7-8 weeks of age were treated with MPTP (b.i.d., 20 mg/kg, s.c.). The mice also receive a daily administration of saline, GM1 (30 mg/kg), or a compound of the invention (0.3 to 3 mg/kg, i.p. and 30 mg/kg os) for three weeks starting 24 h after the last MPTP injection. The brains were removed and analyzed for striatal dopamine levels. The midbrain were fixed for TH immunohistochemistry and dopamine neuron cell counts.

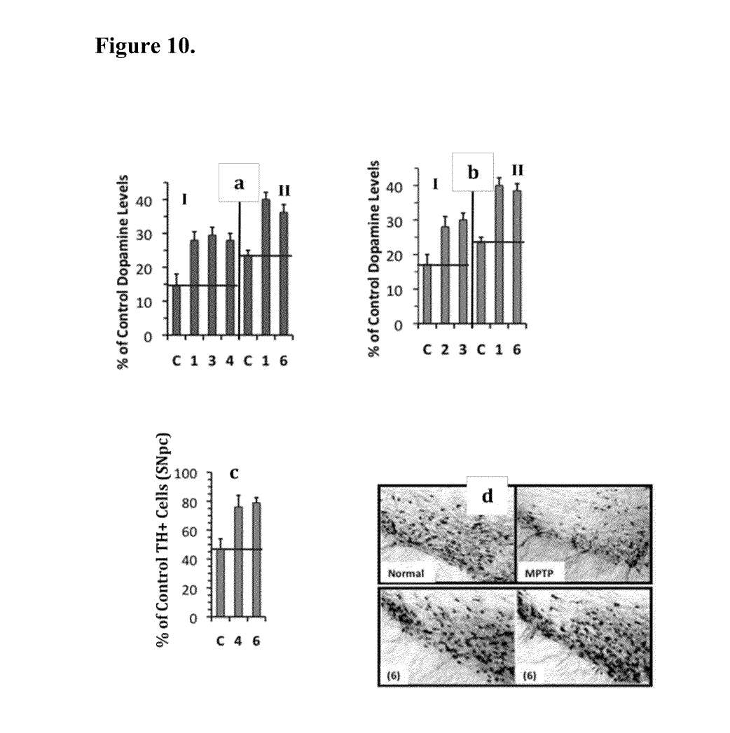

MPTP alone can cause approximately 70% loss of striatal dopamine. GM1 and compounds for use according to the invention increased striatal dopamine levels. The number of TH+ neurons was also determined by staining brain sections in the striatum and substantia nigra (FIG. 10).

### Example 10

### Rescue of Striatal Dopamine Levels and Dopaminergic Cells in the GM1 Deficient Mouse

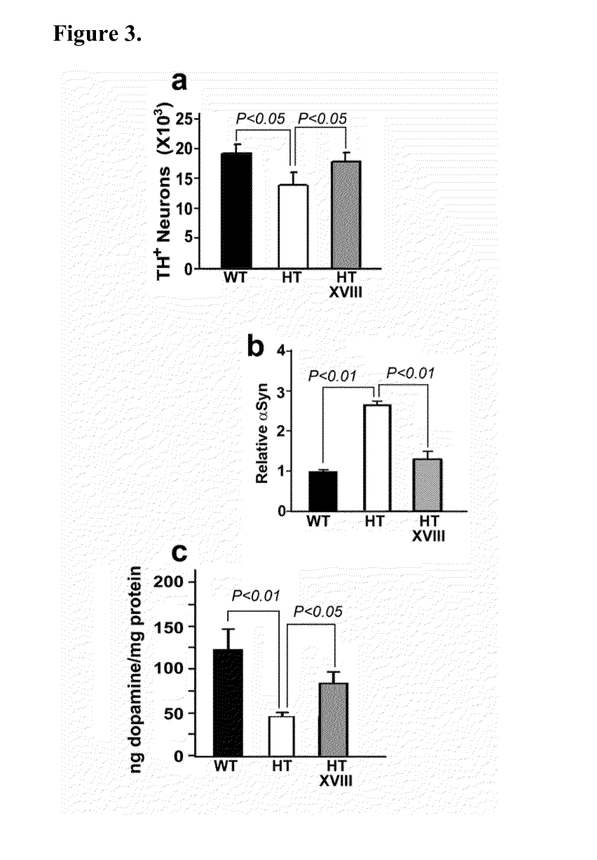

Heterozygote mice (200 days old, DOA) were treated with saline or analog XVIII (2.5 mg/Kg; 3×/wk, IP) over 5 weeks. Immunohistochemistry (IHC) study of the substantia nigra pars compacta (SNpc) revealed increase in TH+ dopaminergic cells, elevation of striatal dopamine, and reduction of αSynuclein (FIG. 3). Similar results were obtained with knock-out (KO) mice, totally deficient in GM 1.

### Example 11

### Improvement of Motor Function in GM1 Deficient Mice

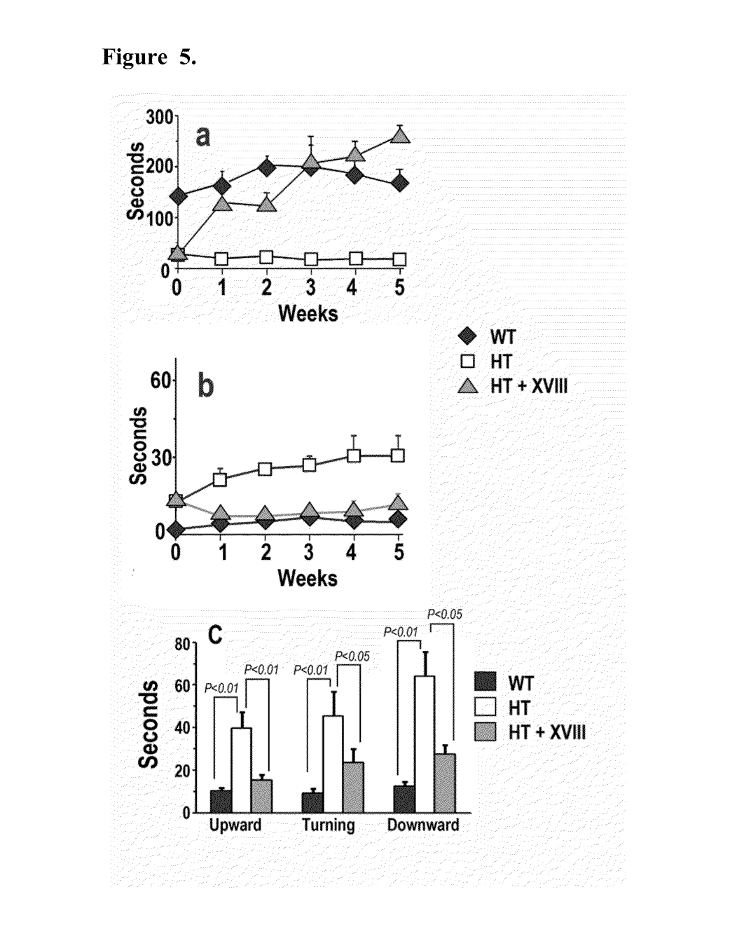

HT mice (200 DOA) were treated with saline or analog XVIII (2.5 mg/Kg; 3×/wk; IP) over 5 weeks. They showed significantly improved motor function as judged by the grip duration, irritant removal, and pole climbing tests (FIG. 5). Similar results were obtained with KO mice.

### Example 12

### Improvement of Cognitive Function in GM1 Deficient Mice

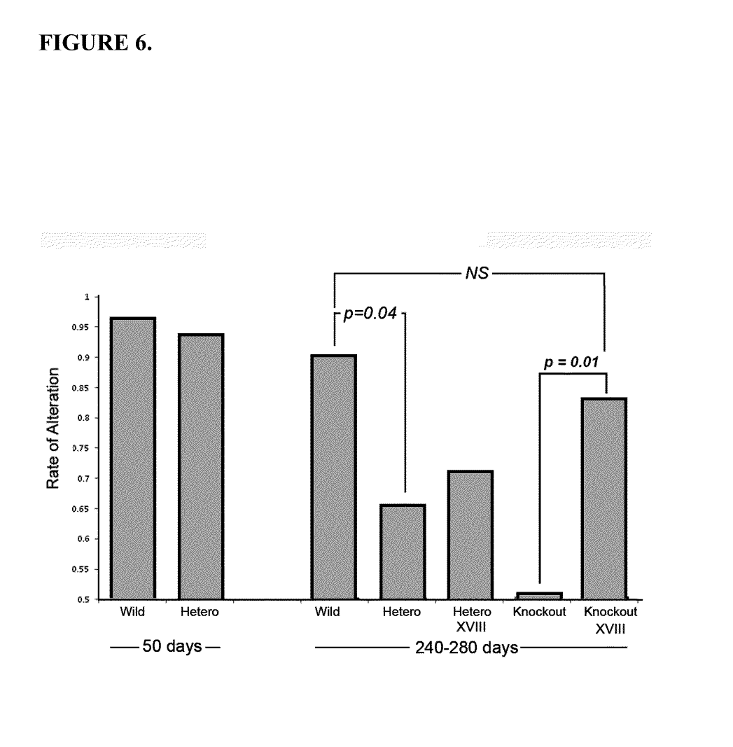

HT and KO mice in two age groups were evaluated for cognitive function with the T maze test (FIG. 6).

### Example 13

### Neurite Outgrowth Assay

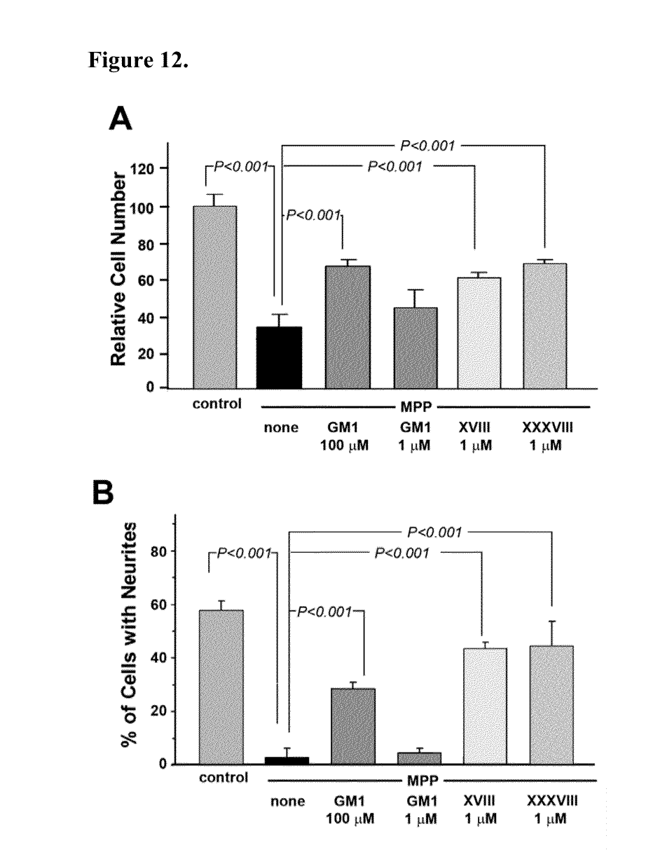

The compounds for use according to the invention can also be identified using the Neurite Outgrowth Assay. Striatal dopanergic neurons in culture were treated with the compounds according to the invention added in various concentrations from 1 μM to 100 μM. The neurite outgrowth effect was observed (FIG. 12).

### Example 14

### Delivery of Compound of Formula (X) to the Brain

A method for brain delivery of compounds of the invention. A patient with idiopathic Parkinson's Disease is treated with compound (X) directly to the brain, using a procedure modified from Gill, S. S., et al., Nat. Med., 9:589-595 (2003). Full consent in accordance with local ethics committees is obtained for the procedure. The patient is analyzed using a mid-sagittal planning PET scan, producing 2-mm thick axial images parallel to the anterior-posterior plane. Coronal images that are orthogonal to the initial images are produced. The patient is placed under general anesthesia, and a stereotactic frame is placed parallel to the orbito-meatal plane. The images are used to target the area of the postero-dorsal putamen with compounds of the invention. 1 mm guide tubes are implanted under stereotaxic conditions to a point above the putamen target over a guide rod. A 0.6 mm guide wire is introduced down the guide tube to the target, and the patient then undergoes repeat magnetic resonance and computed tomography imaging to verify target localization. A primed SynchroMed pump (Medtronic, Minneapolis Minn.) containing compound (X) is implanted in the upper abdominal region, subfascially (beneath the anterior rectus sheath). A catheter is tunneled connecting the pump to the indwelling 0.6 mm intraparenchymal brain catheter.

Compound (X) may be supplied at a concentration between about 500 nM and about 1000 mM, to be pumped in sterile water buffered with hypotonic phosphate buffer. After implantation, the SynchroMed pump is programmed to deliver a continuous infusion at a predetermined concentration, for example, between about 500 nM and about 500 mM, or in a particular embodiment, between about 1 μM and about 500 μM, of compound (X) to the putamen per day at a rate of 6 μL/hr. Clinical evaluations are performed prior to infusion, at 3 month, at 6 months, at 9 months, and at 12 months, using CAPIT (Langston, J. W., et al., Mov. Disord., 7:2-13 (1992)), studying mobility, activities of daily living, emotional wellbeing, stigma, social support, cognition, communication, and bodily discomfort.

### Example 15

### Peripheral Delivery of Compounds

A patient with idiopathic Parkinson's Disease is treated with compound (XXVIII) via once a day subcutaneous injection. Full consent in accordance with local ethics committees is obtained for the procedure. Compound (XXXVIII) may be supplied at 20-100 mg/mL concentration in sterile water buffered with phosphate buffer, pH 7.0. Clinical evaluations are performed prior to the first injection, at 3 month, at 6 months, at 9 months, and at 12 months, using CAPIT (Langston, J. W., et al., Mov. Disord., 7:2-13 (1992)), studying mobility, activities of daily living, emotional wellbeing, stigma, social support, cognition, communication, and bodily discomfort.

Multiple patients (60) are divided into three test groups (placebo, low dose glycolipid, and high dose glycolipid) and administered a glycolipid subcutaneously or orally on a daily basis over a period of from 6 months to two years. Clinical evaluations are performed prior to the first injection, at 3 month, at 6 months, at 9 months, and at 12 months, using CAPIT Langston, J. W., et al., Mov. Disord., 7:2-13 (1992)), studying mobility, activities of daily living, emotional wellbeing, stigma, social support, cognition, communication, and bodily discomfort.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes to the extent not inconsistent with the present disclosure.

What is claimed is:

1. A method of treating a disease or disorder mediated by a GM1 deficiency in a mammal comprising administering to the mammal an effective amount of a compound of the formula: 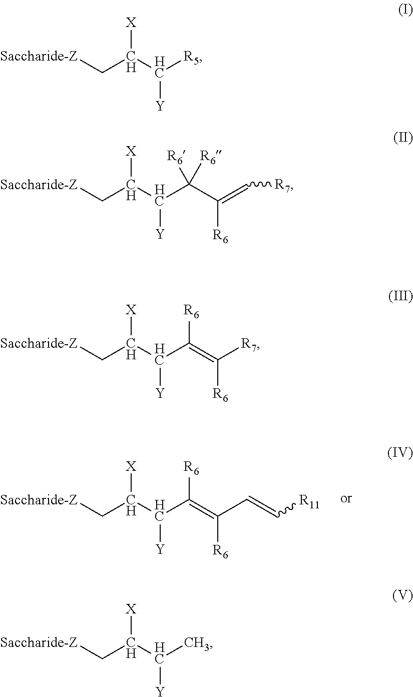
wherein
X is H, D, —OR₃, —NR₃R₄, —SR₃ or —CHR₃R₄;
Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;
Z is O, S or NR₆;
R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and
M is O, S or NR₆.

2. A method of increasing cognitive or motor function or decreasing cognitive or motor function decline a disease or disorder mediated by a GM1 deficiency in a mammal comprising administering to the mammal an effective amount of a compound of the formula: 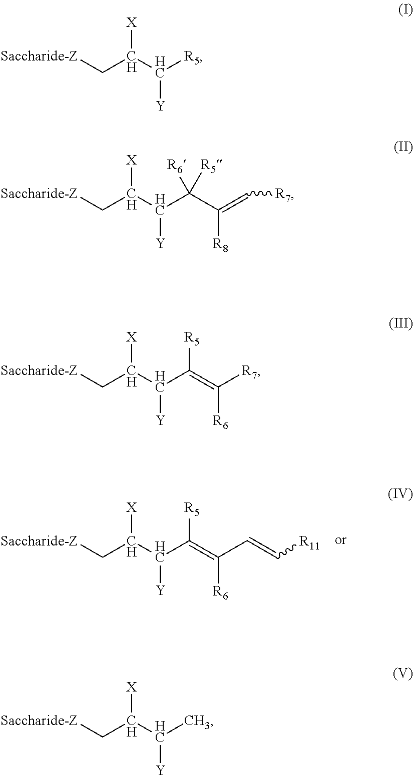
wherein
X is H, D, —OR₃, —NR₃R₄, —SR₃ or —CHR₃R₄;
Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;
Z is O, S or NR₆;
R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and
M is O, S or NR₆.

3. A method of treating Parkinson's disease mediated by GM1 deficiency in a mammal comprising administering to the mammal an effective amount of a compound of the formula: 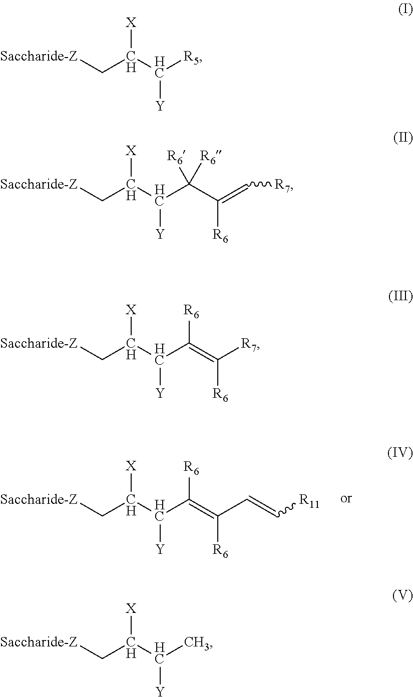
wherein
X is H, D, —OR₃, —NR₃R₄, —SR₃ or —CHR₃R₄;
Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;
Z is O, S or NR₆;
R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and
M is O, S or NR₆.

4. A method of preventing neuronal cell death comprising contacting said neuronal cell that is deficient in GM1 with an effective amount of a compound of the formula: 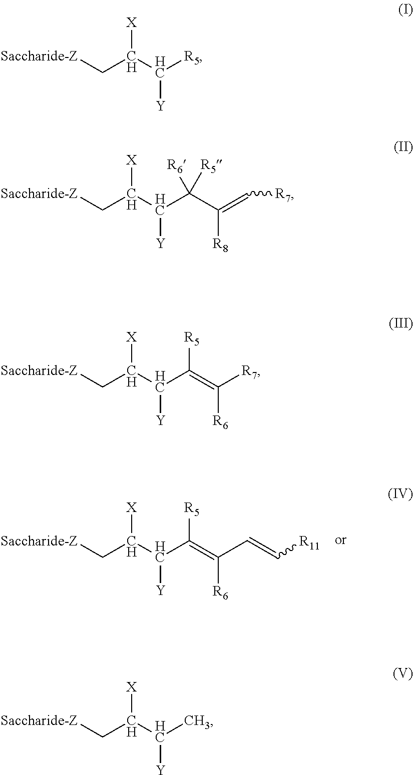
wherein
X is H, D, —OR₃, —NR₃R₄, —SR₃ or —CHR₃R₄;
Y is H, —OR⁷, SR⁷, —NR⁷R⁸, substituted or unsubstituted alkyl, substituted or unsubstituted heteroalkyl, substituted or unsubstituted aryl, substituted or unsubstituted heteroaryl, or substituted or unsubstituted heterocycloalkyl;
Z is O, S or NR₆;
R₁, R₂ and R₃ are independently H, D, substituted or unsubstituted alkyl, substituted or unsubstituted heteroalkyl, substituted or unsubstituted aryl, substituted or unsubstituted heteroaryl, substituted or unsubstituted heterocycloalkyl, —C(=M)R₅, —C(=M)-Z—R₅, —SO₂R₅, or —SO₃;
R₄ is H, D, alkyl, cycloalkyl, aryl, arylalkyl, heteroaryl, heteroalkyl, or haloalkyl;
R₅ is H, D, alkyl, cycloalkyl, alkenyl, aryl, arylalkyl, heteroaryl, heteroalkyl, or haloalkyl;
R₆, R_{6′}, R_{6″}, R₇, R₈ and R₁₁ are independently H, D, substituted or unsubstituted alkyl, substituted or unsubstituted heteroalkyl, substituted or unsubstituted aryl, substituted or unsubstituted heteroaryl, or substituted or unsubstituted heterocycloalkyl; and
M is O, S or NR₆.

5. The method of claim 4, wherein said neuron is a catecholaminergic neuron.

6. The method of claim 4, wherein said neuron is a dopaminergic neuron.

7. The method of claim 1, wherein the saccharide is 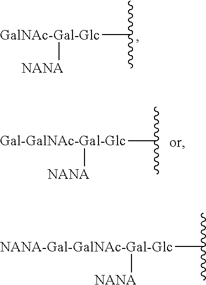
wherein said saccharide is deacylated or not deacylated.

8. The method of claim 2, wherein the saccharide is 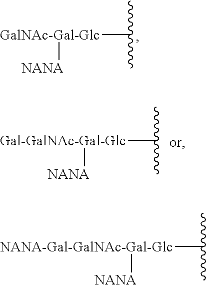
wherein said saccharide is deacylated or not deacylated.

9. The method of claim 3, wherein the saccharide is 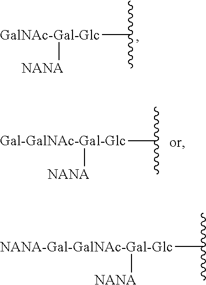
wherein said saccharide is deacylated or not deacylated.

10. The method of claim 4, wherein the saccharide is 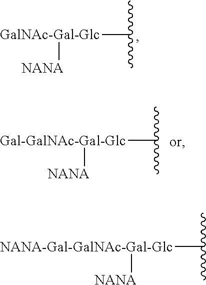
wherein said saccharide is deacylated or not deacylated.

11. The method of claim 1, wherein the saccharide has a formula Galβ(1,3)GalNAcβ(1,4)[NANAα(2,3)]Galβ(1,4)Glc−, wherein NANA is N-acetyl neuraminic acid.

12. The method of claim 2, wherein the saccharide has a formula Galβ(1,3)GalNAcβ(1,4)[NANAα(2,3)]Galβ(1,4)Glc−, wherein NANA is N-acetyl neuraminic acid.

13. The method of claim 3, wherein the saccharide has a formula Galβ(1,3)GalNAcβ(1,4)[NANAα(2,3)]Galβ(1,4)Glc−, wherein NANA is N-acetyl neuraminic acid.

14. The method of claim 4, wherein the saccharide has a formula Galβ(1,3)GalNAcβ(1,4)[NANAα(2,3)]Galβ(1,4)Glc−, wherein NANA is N-acetyl neuraminic acid.

15. The method of claim 1 further comprising administering to the cerebrospinal fluid of said mammal.

16. The method of claim 2 further comprising administering to the cerebrospinal fluid of said mammal.

17. The method of claim 3 comprising administering to the cerebrospinal fluid of said mammal.

18. The method of claim 1, wherein said mammal is human.

19. The method of claim 2, wherein said mammal is human.

20. The method of claim 3, wherein said mammal is human.
